(19) United States
(12) Patent Application Publication
(10) Pub. No.: US 2014/0294923 A1
(43) Pub. Date: Oct. 2, 2014

(54) ACTH FOR TREATMENT OF MIGRAINE HEADACHE

(71) Applicant: Questcor Pharmaceuticals, Inc., Hayward, CA (US)

(72) Inventors: Steve CARTT, San Carlos, CA (US); James KNIGHT, Oakland, CA (US)

(73) Assignee: Questcor Pharmaceuticals, Inc., HayWard, CA (US)

(21) Appl. No.: 14/184,586

(22) Filed: Feb. 19, 2014

Related U.S. Application Data
(60) Provisional application No. 61/767,213, filed on Feb. 20, 2013.

(51) Int. Cl. A61K 38/22; A61K 9/00; A61K 45/06

(52) U.S. Cl. CPC A61K 38/22; A61K 45/06; A61K 9/0078; A61K 9/0031; A61K 9/0014; A61K 9/0043
USPC 424/450; 514/9.7; 424/94.67

(57) ABSTRACT

Provided herein are methods of treatment of migraine headaches comprising administration of adrenocorticotropic hormone (ACTH), or fragment, analog, complex or aggregate thereof, or any combination thereof, to an individual in need thereof.

### BACKGROUND OF THE INVENTION

Migraine headache is a chronic neurological disorder characterized by recurrent moderate to severe headaches often in association with a number of autonomic nervous system symptoms. Typically the headache is unilateral, affecting one half of the head and pulsating in nature, lasting from 2 to 72 hours. Chronic migraine is a common and disabling disorder, affecting more than 10% of the population worldwide at some point in life. Despite the widespread use of anti-seizure medications, triptans, tricyclic antidepressants (TCA) and beta blockers, and botulinum toxin (Botox®), there remains a significant number of patients whose migraines are refractory to these agents or who fail on these treatments for migraine, or the patients are unable to tolerate the treatment medications or cannot use them owing to other comorbidities which contraindicate their use.

The triptan class of drug agents is used in the acute treatment of chronic migraine and several members of the class including sumatriptan and frovatriptan are FDA-approved for acute treatment. The mechanism of action of the triptans is attributed to agonist effects on serotonin 5-HT1B and 5-HT1D receptors in cranial blood vessels (causing constriction) and subsequent inhibition of pro-inflammatory neuropeptide release. Triptans may also act on serotonin receptors in nerve endings as well as the blood vessels, causing a decrease in the release of several neuropeptides, including CGRP and substance P. The only approved drug product in the United States for prophylactic (preventive) treatment of chronic migraine is onabotulinum toxin A (Botox®). Other drugs are used “off-label” as prophylaxis, that is to say that they do not have a drug agency approval for use in migraine prophylaxis. Durham et al. (2004) demonstrated that botulinum neurotoxin type A inhibited release of CGRP from rat trigeminal cultures stimulated with KCl or capsaicin and speculated that the possible prophylactic efficacy of botulinum toxin type A, like the acute efficacy of triptans, may be partly attributed to the toxin's inhibitory effect on release of CGRP from trigeminal neurons.

Two different CGRP receptor antagonists reached late stage clinical trials and demonstrated clinical benefit and proof of concept that CGRP is both a mediator of migraine propagation and pain and that CGRP receptors are a valid therapeutic target. (Tepper and Stillman 2008). ACTH has been shown to inhibit the release of CGRP in experimental models (Ulrich-Lai 2001). Systemic administration of ACTH (H.P. Acthar Gel®) potently reduced CRH (corticotropin-releasing hormone) gene expression in amygdala neurons of both adrenalectomized and intact rats in a melanocortin receptor-dependent fashion, providing experimental evidence that ACTH exerts direct effects on the CNS independent of its steroidogenic effects in the adrenal glands (Brunson, et al. 2001).

### SUMMARY OF THE INVENTION

Described herein are methods of preventing or treating an individual diagnosed with migraine (e.g., chronic migraine) comprising administration of an adrenocorticotropic hormone (ACTH) peptide, or fragment, analog, complex or aggregate thereof, or any combination thereof, to an individual in need thereof. In some embodiments, the methods described herein can be used to treat an individual diagnosed with migraine comprising administration of an adrenocorticotropic hormone (ACTH) peptide, or fragment, analog, complex or aggregate thereof, or any combination thereof, to an individual in need thereof at a therapeutically effective amount and interval. The methods described herein can be used to treat various types of migraine headaches including chronic migraine, migraine with aura, migraine without aura, familial hemiplegic migraine, sporadic hemiplegic migraine, basilar-type migraine, status migrainosus, probable migraine and retinal migraine. In some embodiments, the methods described herein are used to treat chronic migraine. In some embodiments, the methods described herein are used to treat status migrainosus. In some embodiments, the treatments described herein are prophylactic and used to treat chronic migraine. In some embodiments, the treatments described herein are acute and used to treat status migrainosus.

In some embodiments, the ACTH peptide or fragment, analog, complex or aggregate thereof, or any combination thereof, is administered as a first dose and one or more subsequent doses.

In some embodiments, the first dose of adrenocorticotropic hormone (ACTH) peptide, or fragment, analog, complex or aggregate thereof, or any combination thereof, to an individual in need thereof is between about 10 IU and about 150 IU.

In some embodiments, the first dose of ACTH peptide or fragment, analog, complex or aggregate thereof, or any combination thereof, comprises a dose between about 10 IU and about 100 IU.

In some embodiments, the first dose of ACTH peptide or fragment, analog, complex or aggregate thereof, or any combination thereof, comprises a dose between about 10 IU and about 150 IU, and the one or more subsequent doses of ACTH peptide or fragment, analog, complex or aggregate thereof, or any combination thereof, are between about 20% and about 80% of the first dose.

In some embodiments, the first dose of ACTH peptide or fragment, analog, complex or aggregate thereof, or any combination thereof, comprises an initial phase of one to five doses at daily intervals of about 30 IU and about 120 IU, and one or more subsequent doses of ACTH peptide or fragment, analog, complex or aggregate thereof, or any combination thereof, are between about 20% and about 100% of the initial dose and at intervals of about every two days, three times per week, two times per week, once a week, once every two weeks to about once per month.

In some embodiments, the first dose of ACTH peptide or fragment, analog, complex or aggregate thereof, or any combination thereof, comprises a first dose of between about 10 IU and about 150 IU, and the one or more subsequent doses of ACTH peptide or fragment, analog, complex or aggregate thereof, or any combination thereof, are between about 20% and about 60% of the first dose.

In some embodiments, the first dose of ACTH peptide or fragment, analog, complex or aggregate thereof, or any combination thereof is between about 30 IU and about 100 IU, and the one or more subsequent doses of ACTH peptide or fragment, analog, complex or aggregate thereof, or any combination thereof, are between about 10 IU and about 80 IU.

In some embodiments, the first dose of ACTH peptide or fragment, analog, complex or aggregate thereof, or any combination thereof is between about 45 IU and about 100 IU, and the one or more subsequent doses of ACTH peptide or fragment, analog, complex or aggregate thereof, or any combination thereof, are between about 10 IU and about 80 IU.

In some embodiments, the first dose of ACTH peptide or fragment, analog, complex or aggregate thereof, or any combination thereof is between about 60 IU and about 100 IU, and the one or more subsequent doses of ACTH peptide or fragment, analog, complex or aggregate thereof, or any combination thereof, are between about 20 IU and about 60 IU.

In some embodiments, the first dose of ACTH peptide or fragment, analog, complex or aggregate thereof, or any combination thereof is between about 80 IU, and the one or more subsequent doses of ACTH peptide or fragment, analog, complex or aggregate thereof, or any combination thereof is about 40 IU.

In some embodiments, one or more subsequent doses of ACTH peptide or fragment, analog, complex or aggregate thereof, or any combination thereof, are administered about every day, about every 2 days, about every 5 days, about every week, about every two weeks, about every three weeks, about every month, about every two months, or any combination thereof.

In another embodiment, the dosage of ACTH peptide or fragment, analog, complex or aggregate thereof, or any combination thereof, comprises from about 40 IU to about 80 IU wherein each dosage is initially once a day for 5 days during the first week of treatment and then dosage frequency is 3 times during each subsequent week, approximately every other day. For example, dosages may be administered on Monday, Wednesday and Friday of each subsequent treatment week.

In some embodiments, the ACTH peptide or fragment, analog, complex or aggregate thereof, or any combination thereof, is administered as an initial phase of one or more doses and a subsequent phase of one or more doses. In some cases, the initial phase is one, two, three, four, five, six, seven, eight, nine, or ten doses. In some cases, the initial phase is from one to two, one to three, one to four, one to five, one to six, one to seven, one to eight, one to nine, or one to ten doses. In some embodiments, the dosing intervals of the initial phase and the subsequent are the same. In some embodiments, the dosing intervals of the initial phase and the subsequent are different. In some embodiments, the ACTH peptide or fragment, analog, complex or aggregate thereof, or any combination thereof, is administered as an initial phase at daily intervals and a subsequent phase at intervals greater than one day. In some embodiments, the one or more of the doses of the subsequent phase are administered at intervals of every other day or once every two days, once every three days, once every four days, once every five days, once every six days, once a week, twice a week, three times a week, four times a week, five times a week, six times a week, or once every two weeks, once every three weeks, once every four weeks, once every month, one every two months, or once every three months. In some embodiments, the ACTH peptide or fragment, analog, complex or aggregate thereof, or any combination thereof, is administered as an initial phase of from one to five doses at daily intervals and a subsequent phase of one or more doses administered at intervals greater than one day. In some embodiments, each of the one to five doses of the initial phase is between about 10 IU and about 150 IU. In some embodiments, each of the one to five doses of the initial phase is between about 30 IU and about 100 IU. In some embodiments, each of the one to five doses of the initial phase is between about 30 IU and about 100 IU, and each of the one or more doses of the subsequent phase is between about 20% and about 100% of the one to five doses of the initial phase. In some embodiments, each of the one to five doses of the initial phase is between about 10 IU and about 150 IU, and each of the one or more doses of the subsequent phase is between about 10 IU and about 80 IU. In some embodiments, each of the one to five doses of the initial phase is between about 45 IU and about 100 IU, and each of the one or more doses of the subsequent phase is between about 10 IU and about 80 IU. In some embodiments, each of the one to five doses of the initial phase is between about 60 IU and about 100 IU, and each of the one or more doses of the subsequent phase is between about 20 IU and about 60 IU. In some embodiments, each of the one to five doses of the initial phase is about 80 IU, and each of the one or more doses of the subsequent phase is about 40 IU.

In the foregoing embodiments, the dosage may be titrated up or down within the stated ranges depending on the patient's response to therapy or appearance of side effects.

In some embodiments, the ACTH peptide or fragment, analog, complex or aggregate thereof, or any combination thereof, is an ACTH₁₋₃₉ peptide having the formula:

(SEQ ID NO: 1)
H -Ser-Tyr-Ser-Met-Glu-His-Phe-Arg-Trp-Gly-
1 2 3 4 5 6 7 8 9 10
Lys-Pro-Val-Gly-Lys-Lys-Arg-Arg-Pro-Val-
11 12 13 14 15 16 17 18 19 20
Lys-Val-Tyr-Pro-Asp-Gly-Ala-Glu-Asp-Gln-
21 22 23 24 25 26 27 28 29 30
Leu-Ala-Glu-Ala-Phe-Pro-Leu-Glu-Phe-OH
31 32 33 34 35 36 37 38 39

or a fragment, complex, aggregate, or analog thereof, or any combination thereof.

In some embodiments, the ACTH peptide or fragment, analog, complex or aggregate thereof, or any combination thereof, is an ACTH₁₋₂₄ peptide having the formula:

(SEQ ID NO: 2)
H -Ser-Tyr-Ser-Met-Glu-His-Phe-Arg-Trp-Gly-
1 2 3 4 5 6 7 8 9 10
Lys-Pro-Val-Gly-Lys-Lys-Arg-Arg-Pro-Val-
11 12 13 14 15 16 17 18 19 20
Lys-Val-Tyr-Pro
21 22 23 24

or a fragment, complex, aggregate, or analog thereof, or any combination thereof.

In some embodiments, the ACTH peptide or fragment, analog, complex or aggregate thereof, or any combination thereof, is an ACTH₁₋₁₇ peptide having the formula:

(SEQ ID NO: 3)
H -Ser-Tyr-Ser-Met-Glu-His-Phe-Arg-Trp-Gly-
1 2 3 4 5 6 7 8 9 10
Lys-Pro-Val-Gly-Lys-Lys-Arg
11 12 13 14 15 16 17

or a fragment, complex, aggregate, or analog thereof, or any combination thereof.

In some embodiments, the ACTH peptide or fragment, analog, complex or aggregate thereof, or any combination thereof, is an ACTH₄₋₁₀ peptide of formula:

(SEQ ID NO: 4)
Met-Glu-His-Phe-Arg-Trp-Gly
4 5 6 7 8 9 10

or a fragment, complex, aggregate, or analog thereof, or any combination thereof.

In some embodiments, the ACTH peptide or fragment, analog, complex or aggregate thereof, or any combination thereof, is an ACTH₄₋₉ peptide analog of formula:

(SEQ ID NO: 5)
O₂-Met-Glu-His-Phe-D-Lys-Phe-OH

or a fragment, complex, aggregate, or analog thereof, or any combination thereof.

In some embodiments, the ACTH peptide or fragment, analog, complex or aggregate thereof, or any combination thereof, is a fragment of formula:

(SEQ ID NO: 6)
Ac-Ser-Tyr-Ser-Met-Glu-His-Phe-Arg-Trp-Gly-Lys-
Pro-Val-NH₂

or a fragment, complex, aggregate, or analog thereof, or any combination thereof.

In some embodiments, the ACTH peptide or fragment, analog, complex or aggregate thereof, or any combination thereof, is a fragment of formula

(SEQ ID NO: 7)
H-Ser-Tyr-Ser-Met-Glu-His-Phe-Arg-Trp-Gly-Lys-
Pro-Val

or a fragment, complex, aggregate, or analog thereof, or any combination thereof.

In some embodiments, the ACTH peptide or fragment, analog, complex or aggregate thereof, or any combination thereof, is a fragment of formula

(SEQ ID NO: 8)
D-Ala-Gln-Tyr-Phe-Arg-Trp-Gly-NH₂

or a fragment, complex, aggregate, or analog thereof, or any combination thereof.

In some embodiments, the ACTH peptide or fragment, analog, complex or aggregate thereof, or any combination thereof, is a fragment peptide chosen from the group of:

(SEQ ID NO: 3)
H -Ser-Tyr-Ser-Met-Glu-His-Phe-Arg-Trp-Gly-
Lys-Pro-Val-Gly-Lys-Lys-Arg;
(SEQ ID NO: 4)
Met-Glu-His-Phe-Arg-Trp-Gly;
(SEQ ID NO: 5)
O₂-Met-Glu-His-Phe-D-Lys-Phe-OH;
(SEQ ID NO: 6)
Ac-Ser-Tyr-Ser-Met-Glu-His-Phe-Arg-Trp-Gly-Lys-
Pro-Val-NH₂;
(SEQ ID NO: 7)
H-Ser-Tyr-Ser-Met-Glu-His-Phe-Arg-Trp-Gly-Lys-
Pro-Val; and
(SEQ ID NO: 8)
D-Ala-Gln-Tyr-Phe-Arg-Trp-Gly-NH₂;

or a complex, aggregate, pharmaceutically acceptable salt, ester or amide thereof, or any combination thereof.

In some embodiments, the ACTH peptide or fragment, analog, complex or aggregate thereof, or any combination thereof is a peptide of 6-10 amino acids, 11-15 amino acids, 16-20 amino acids, 21-25 amino acids, 26-30 amino acids, 31-35 amino acids, 36-40 amino acids or 40-45 amino acids.

In some embodiments, the ACTH peptide or fragment, analog, complex or aggregate thereof, or any combination thereof is a peptide with about 50%, 60%, 70%, 80%, 90%, or 100% homology to ACTH.

In some embodiments the ACTH peptide or fragment, analog, complex or aggregate thereof, or any combination thereof, is administered as an ACTH formulation. Accordingly, in some embodiments, ACTH formulations include, but are not limited, to an aerosol, nasal spray, syrup, gel, liquid solution, emulsion, suspension, powder, injectable solution, aqueous liquid dispersion, self-emulsifying dispersion, solid solution, liposomal dispersion, immediate release formulation, prodrug, prolonged release formulation, controlled release formulation, fast melt formulation, delayed release formulation, pulsatile release formulation, multiparticulate formulation or mixed immediate and controlled release formulation. In some embodiments, the ACTH peptide or fragment, analog, complex or aggregate thereof, or any combination thereof, is administered as a prodrug. In some embodiments, the ACTH peptide or fragment, analog, complex or aggregate thereof, or any combination thereof, is administered as a prolonged release formulation. In some embodiments, the ACTH peptide or fragment, analog, complex, or aggregate thereof, or any combination thereof, is administered as a nasal spray. In some embodiments, the ACTH peptide or fragment, analog, complex or aggregate thereof, or any combination thereof, is formulated as a gel.

In some embodiments, the ACTH peptide or fragment, analog, complex or aggregate thereof, or any combination thereof, is administered intramuscularly, subcutaneously, intravenously, intrathecally, orally, systemically, topically, transmucosally, parenterally, intranasally, bucally, rectally, transdermally, by inhalation or by implant. In some embodiments, the ACTH peptide or fragment, analog, complex or aggregate thereof, or any combination thereof, is administered intramuscularly. In some embodiments, the ACTH peptide or fragment, analog, complex or aggregate thereof, or any combination thereof, is administered subcutaneously. In some embodiments, the ACTH peptide or fragment, analog, complex or aggregate thereof, or any combination thereof, is administered intravenously. In some embodiments, the ACTH peptide or fragment, analog, complex or aggregate thereof, or any combination thereof, is administered intrathecally. In some embodiments, the ACTH peptide or fragment, analog, complex or aggregate thereof, or any combination thereof, is administered with an implant. In some embodiments, the ACTH peptide or fragment, analog, complex or aggregate thereof, or any combination thereof, is administered via an erodible implant.

In some embodiments of the methods described herein, the ACTH peptide or fragment, analog, complex or aggregate thereof, or any combination thereof, is administered as an ACTH preparation. For example, in some embodiments, the ACTH peptide or fragment, analog, complex or aggregate thereof, or any combination thereof, is a porcine, human, bovine, sheep, rabbit, chicken, goat or recombinant ACTH peptide or fragment, analog, complex or aggregate thereof, or any combination thereof. In some embodiments, the ACTH peptide or fragment, analog, complex or aggregate thereof, or any combination thereof, is a porcine ACTH peptide or fragment, analog, complex or aggregate thereof, or any combination thereof. In some embodiments, the ACTH peptide or fragment, analog, complex or aggregate thereof, or any combination thereof, is a human ACTH peptide or fragment, analog, complex or aggregate thereof, or any combination thereof. In some embodiments, the ACTH peptide or fragment, analog, complex or aggregate thereof, or any combination thereof, is a recombinant ACTH peptide or fragment, analog, complex or aggregate thereof, or any combination thereof. In some embodiments, the ACTH peptide or fragment, analog, complex or aggregate thereof, or any combination thereof, is a recombinant human ACTH peptide or fragment, analog, complex or aggregate thereof, or any combination thereof.

In some embodiments, the methods described herein are used to treat or prevent a migraine headache that is refractory or does not respond adequately to other migraine treatments. Such other migraine treatments include, but are not limited to: antiepileptics, including topiramate gabapentin, pregabalin, valproate sodium, and zonisamide; onabotulinum toxin A (Botox®, Dysport®) and botulinum toxin B (Neurobloc®, Myobloc®), tizanidine, tricyclic antidepressants (TCA's), including amitriptyline, nortriptyline, protriptyline, doxepin and imipramine; fluoxetine, memantine, sumatriptan, dihydroergotamine (DHE, Migranal®, Levadex®), metoclopramide, hydroxyzine, 5-HT2 antagonists including the ergots, methysergide, methylergonovine and methylergometrine, neuroleptics, including prochlorperazine, chlorpromazine and droperidol, corticosteroids, magnesium, ketorolac, acetaminophen, opioids, antiemetics (metoclopramide, ondansetron), vitamin B12, timolol, or propranolol.

In some embodiments, the methods described herein further comprise administration of a second therapeutic agent. In some embodiments, the second therapeutic agent is administered sequentially or simultaneously. The second therapeutic agent can be, for example, antiepileptics, including topiramate gabapentin, pregabalin, valproate sodium, and zonisamide; onabotulinumtoxin A (Botox®, Dysport®) and botulinum toxin B (Neurobloc®, Myobloc®), tizanidine, tricyclic antidepressants (TCA's), including amitriptyline, imipramine, nortriptyline, protriptyline and doxepin; fluoxetine; memantine; triptans, including sumatriptan (Imitrex®, Treximet®), frovatriptan (Frova®), almotriptan (Axert®), rizatriptan (Maxalt®), zolmitriptan (Zomig®); dihydroergotamine (DHE, Migranal®, Levadex®), metoclopramide, hydroxyzine; ergot derivatives, including methysergide, methylergonovine and methylergometrine; neuroleptics, including prochlorperazine, chlorpromazine and droperidol; corticosteroids; magnesium; NSAIDS, including ketorolac, naproxen sodium and acetaminophen; opioids, antiemetics (metoclopramide, ondansetron), vitamin B12, timolol or propranolol.

In some embodiments, the methods are directed to headache preventive or prophylactic therapy, such as for chronic migraine headache. In another embodiment, the methods of chronic treatment are for a patient who experiences daily migraine episodes. In some embodiments, the methods are used when a patient's headaches are refractory to onabotulinum toxin A (Botox®).

In some embodiments the methods described herein are used in acute treatment of a migraine headache, including treatment of status migrainosus. In some embodiments, the methods described herein provide treatment options to patients in whom triptans and ergotamines are contraindicated due to cardiovascular disease, coronary artery disease, cerebrovascular disease, peripheral vascular disease, hypertension, other concomitant medications or other conditions.

In some embodiments, the methods described herein can be used to treat migraine headaches associated with one or more comorbidities. These comorbidities include, but are not limited to, anxiety, depression, obesity, medication overuse, infections, trauma, hyperexcitability of pain systems, abnormal levels or activity of neurotransmitters, sinus, oral, or dental pathologies, cervical or other kinds of spinal injury, and abnormal iron and electrolyte levels. In some embodiments, medications overused include but are not limited to analgesics, decongestants, opioids, bualbital combinations, isometheptene, benzodiazepines, ergotamine tartrate, triptans, barbiturates and combinations thereof.

In some embodiments, the methods described herein may be used for treatment of migraine headaches associated with other types of headaches. These other types of headaches include but are not limited to a tension-type headache, cluster headache, hemicrania continua, stabbing headache, cough headache, exertional headache, headache associated with sexual activity, hynic headache, thunderscap headache, new daily-persistent headache, headache attributed to neck or head trauma, headache attributed to cranial or cervical vascular disorder, headache attributed to non-vascular intracranial disorder, headache attributed to a substance or its withdrawal, headache attributed to infection, headache attributed to disorder of homeostasis, and a headache attributed to a psychiatric disorder.

In some embodiments, the methods described herein may be used for the treatment of an individual diagnosed with migraine wherein the individual's migraine is refractory to other migraine treatments. In some embodiments, the methods described herein may be used for the treatment of an individual diagnosed with migraine wherein the individual's migraine is refractory to onabotulinum toxin A (Botox®) treatment. In some embodiments, the methods described herein may be used for the treatment of an individual diagnosed with migraine wherein the individual is contraindicated for treatment with another migraine medication. In further embodiments, the contraindicated treatment is chosen from triptans, and an ergot derivative, including dihydroergotamine (DHE, Migranal®, Levadex®), methysergide, methylergonovine and methylergometrine.

In addition, described herein are methods of reducing one or more side effects that occur with the administration of an adrenocorticotropic hormone (ACTH) peptide, or fragment, analog, complex or aggregate thereof, or any combination thereof, to an individual in need thereof. This can be accomplished, for example, by titration of the dosage (e.g., the dosage can be titrated up or down). Side effects that can be reduced include weight gain, anxiety, palpitations, dizziness, pain in extremities, general discomfort, fatigue, edemas, itching, thirst, aphonia, hypermenorrhea, hot flashes, nausea, vomiting, concentration difficulties and irritability.

In further embodiments, the methods described herein comprise administration of an adrenocorticotropic hormone (ACTH) peptide, or fragment, analog, complex or aggregate thereof, or any combination thereof, to an individual in need thereof, wherein the ACTH peptide or fragment, analog, complex or aggregate thereof, or any combination thereof is administered as a first dose and one or more subsequent doses.

In further embodiments, the methods described herein comprise administration of adrenocorticotropic hormone (ACTH) peptide, or fragment, analog, complex or aggregate thereof, or any combination thereof, to an individual in need thereof, wherein the ACTH peptide or fragment, analog, complex or aggregate thereof, or any combination thereof, is an ACTH₁₋₃₉ peptide having the formula:

(SEQ ID NO: 1)
H -Ser-Tyr-Ser-Met-Glu-His-Phe-Arg-Trp-Gly-
1 2 3 4 5 6 7 8 9 10
Lys-Pro-Val-Gly-Lys-Lys-Arg-Arg-Pro-Val-
11 12 13 14 15 16 17 18 19 20
Lys-Val-Tyr-Pro-Asp-Gly-Ala-Glu-Asp-Gln-
21 22 23 24 25 26 27 28 29 30
Leu-Ala-Glu-Ala-Phe-Pro-Leu-Glu-Phe-OH
31 32 33 34 35 36 37 38 39

or a fragment, complex, aggregate, or analog thereof, or any combination thereof.

In further embodiments, the methods described herein comprise administration of an ACTH peptide or fragment, analog, complex or aggregate thereof, or any combination thereof, wherein the ACTH peptide or fragment, analog, complex or aggregate thereof, or any combination thereof, is an ACTH₁₋₃₉ peptide having the formula:

```
(SEQ ID NO: 1)
H-Ser-Tyr-Ser-Met-Glu-His-Phe-Arg-Trp-Gly-
      1 2 3 4 5 6 7 8 9 10
Lys-Pro-Val-Gly-Lys-Lys-Arg-Arg-Pro-Val-
11 12 13 14 15 16 17 18 19 20
Lys-Val-Tyr-Pro-Asp-Gly-Ala-Glu-Asp-Gln-
  21 22 23 24 25 26 27 28 29 30
Leu-Ala-Glu-Ala-Phe-Pro-Leu-Glu-Phe-OH
  31 32 33 34 35 36 37 38 39
```

as a first dose and one or more subsequent doses.

In further embodiments, the methods described herein comprise administration of an ACTH peptide or fragment, analog, complex or aggregate thereof, or any combination thereof as a first and one or more subsequent doses, wherein the first dose of ACTH peptide or fragment, analog, complex or aggregate thereof, or any combination thereof is about 80 IU, and the one or more subsequent doses of ACTH peptide or fragment, analog, complex or aggregate thereof, or any combination thereof is about 40 IU.

In further embodiments, the methods described herein comprise administration of an ACTH₁₋₃₉ peptide or fragment, analog, complex or aggregate thereof, or any combination thereof, having the formula:

(SEQ ID NO: 1)
H-Ser-Tyr-Ser-Met-Glu-His-Phe-Arg-Trp-Gly-
1 2 3 4 5 6 7 8 9 10
Lys-Pro-Val-Gly-Lys-Lys-Arg-Arg-Pro-Val-
11 12 13 14 15 16 17 18 19 20
Lys-Val-Tyr-Pro-Asp-Gly-Ala-Glu-Asp-Gln-
21 22 23 24 25 26 27 28 29 30
Leu-Ala-Glu-Ala-Phe-Pro-Leu-Glu-Phe-OH
31 32 33 34 35 36 37 38 39

as a first and one or more subsequent doses, wherein the first dose is about 80 IU, and the one or more subsequent doses is about 40 IU.

In further embodiments, the methods described herein comprise a method of preventing or treating an individual diagnosed with chronic migraine with an adrenocorticotropic hormone (ACTH) peptide, or fragment, analog, complex or aggregate thereof, or any combination thereof, to an individual in need thereof.

In some embodiments, the adrenocorticotropic hormone (ACTH) peptide, or fragment, analog, complex or aggregate thereof is administered to an individual diagnosed with chronic migraine, as a first dose and one or more subsequent doses.

Described herein is a method of preventing or treating an individual diagnosed with chronic migraine comprising administration of adrenocorticotropic hormone (ACTH) peptide, or fragment, analog, complex or aggregate thereof, or any combination thereof, wherein the ACTH peptide or fragment, analog, complex or aggregate thereof, or any combination thereof is administered as a first dose and one or more subsequent doses.

Described herein is the use of adrenocorticotropic hormone (ACTH) peptide, or fragment, analog, complex or aggregate thereof, or any combination thereof for treating an individual diagnosed with chronic migraine wherein the ACTH peptide or fragment, analog, complex or aggregate thereof, or any combination thereof, is administered as a first dose and one or more subsequent doses.

In further embodiments, the methods described herein may be used for preventing or treating an individual diagnosed with chronic migraine comprising administration of an ACTH peptide or fragment, analog, complex or aggregate thereof, or any combination thereof, wherein the ACTH is an ACTH₁₋₃₉ peptide having the formula:

```
(SEQ ID NO: 1)
H-Ser-Tyr-Ser-Met-Glu-His-Phe-Arg-Trp-Gly-
      1 2 3 4 5 6 7 8 9 10
Lys-Pro-Val-Gly-Lys-Lys-Arg-Arg-Pro-Val-
11 12 13 14 15 16 17 18 19 20
Lys-Val-Tyr-Pro-Asp-Gly-Ala-Glu-Asp-Gln-
  21 22 23 24 25 26 27 28 29 30
Leu-Ala-Glu-Ala-Phe-Pro-Leu-Glu-Phe-OH
  31 32 33 34 35 36 37 38 39
```

or a fragment, complex, aggregate, or analog thereof, or any combination thereof.

In further embodiments, the methods described herein may be used for preventing or treating an individual diagnosed with chronic migraine comprising administration of an ACTH peptide or fragment, analog, complex or aggregate thereof, or any combination thereof, wherein the ACTH is an ACTH₁₋₃₉ peptide having the formula:

```
(SEQ ID NO: 1)
H-Ser-Tyr-Ser-Met-Glu-His-Phe-Arg-Trp-Gly-
      1 2 3 4 5 6 7 8 9 10
Lys-Pro-Val-Gly-Lys-Lys-Arg-Arg-Pro-Val-
11 12 13 14 15 16 17 18 19 20
Lys-Val-Tyr-Pro-Asp-Gly-Ala-Glu-Asp-Gln-
  21 22 23 24 25 26 27 28 29 30
Leu-Ala-Glu-Ala-Phe-Pro-Leu-Glu-Phe-OH
31 32 33 34 35 36 37 38 39
```

as a first dose and one or more subsequent doses.

In some embodiments, the methods described herein may be used for preventing or treating an individual diagnosed with chronic migraine comprising administration of an ACTH peptide or fragment, analog, complex or aggregate thereof, or any combination thereof as a first and one or more subsequent doses, wherein the first dose of ACTH peptide or fragment, analog, complex or aggregate thereof, or any combination thereof is about 80 IU, and the one or more subsequent doses of ACTH peptide or fragment, analog, complex or aggregate thereof, or any combination thereof is about 40 IU.

In some embodiments, the methods described herein may be used for preventing or treating an individual diagnosed with chronic migraine comprising administration of an ACTH₁₋₃₉ peptide or fragment, analog, complex or aggregate thereof, or any combination thereof, having the formula:

```
(SEQ ID NO: 1)
H-Ser-Tyr-Ser-Met-Glu-His-Phe-Arg-Trp-Gly-
      1 2 3 4 5 6 7 8 9 10
Lys-Pro-Val-Gly-Lys-Lys-Arg-Arg-Pro-Val-
11 12 13 14 15 16 17 18 19 20
Lys-Val-Tyr-Pro-Asp-Gly-Ala-Glu-Asp-Gln-
  21 22 23 24 25 26 27 28 29 30
Leu-Ala-Glu-Ala-Phe-Pro-Leu-Glu-Phe-OH
  31 32 33 34 35 36 37 38 39
```

as a first and one or more subsequent doses, wherein the first dose is about 80 IU, and the one or more subsequent doses is about 40 IU.

In addition, described herein are methods of preventing or treating an individual diagnosed with migraine (e.g., chronic migraine) comprising administration of a γ-MSH peptide, or fragment, analog, complex or aggregate thereof, or any combination thereof, to an individual in need thereof.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### DETAILED DESCRIPTION OF THE INVENTION

Provided herein, in some embodiments, are methods of treatment of migraine headache comprising administration of ACTH to an individual in need thereof. Without being bound by theory, it is thought that the pathophysiology of migraine is such that the neural substances (neuropeptides) calcitonin G related protein (CGRP), substance P, and neurokinin A are released at the trigeminal nerve endings innervating the large cranial and dura mater blood vessels, and these substances lead to or facilitate neurotransmission which generates migraine associated pain. Because of this, migraine treatment can be directed to down regulating those neural substances or blocking their receptor sites accordingly. In addition, migraine pain may be relieved by inhibiting CRH release and thus inhibiting mast cell degranulation which leads to vascular dilation and release of other pro-inflammatory mediators which are implicated in migraine.

ACTH is a hormone that is secreted by the pituitary gland and is a part of the hypothalamus-pituitary-adrenal (HPA) axis that maintains the stress response and homeostasis in the body. In some instances ACTH plays a role in neuronal function. Thus, modulation of levels of ACTH can have significant physiological implications.

Advantageously, in some embodiments, the methods of treatment of migraine headache (e.g., chronic migraine) described herein comprise administration of adrenocorticotropic hormone (ACTH) to an individual in need thereof in doses and/or dosing regimens such that, for example, any dysregulation in the production or activity of neuropeptides (e.g. CGRP) known to be involved in migraine headache is remedied or partially remedied, thereby alleviating the symptoms of migraine headache. In certain embodiments, the doses and/or dosing regimens described herein are designed to minimize any abrupt shifts in ACTH levels in an individual (e.g., a surge, or a drop in levels of ACTH).

In some embodiments, the methods of treatment of migraine headache described herein alleviate symptoms in patients that do not obtain relief from anti-seizure medications, TCAs, beta blockers or botulinum toxins.

In some embodiments, the methods described herein are designed to minimize at least one side effect (e.g. weight gain, anxiety, nausea) that occurs with the administration of adrenocorticotropic hormone (ACTH) peptide, or fragment, analog, complex or aggregate thereof, or any combination thereof, for the treatment of migraine headache to an individual in need thereof, e.g., by titration of the dosage.

### Migraine Headaches

Migraine headache is a chronic neurological disorder characterized by recurrent moderate to severe headaches often in association with a number of autonomic nervous system symptoms. Typically the headache is unilateral (affecting one half of the head) and pulsating in nature, lasting from 2 to 72 hours. Associated symptoms can include nausea, vomiting, photophobia (increased sensitivity to light), phonophobia (increased sensitivity to sound) and the pain is generally aggravated by physical activity. Up to one-third of people with migraine headaches perceive an aura: a transient visual, sensory, language, or motor disturbance which signals that the headache will soon occur.

Migraines are divided into several subclasses including migraine without aura, migraine with aura or “classic migraine,” which includes familial hemiplegic migraine and sporadic hemiplegic migraine, abdominal migraine, retinal migraine and chronic migraine. Chronic migraine is typically defined by a headache (e.g., a tension-type and/or migraine) that is experienced fifteen or more days per month for at least one month or at least two months or at least three months, wherein eight or more of those fifteen days per month the headache is migrainous in nature. The eight or more migrainous headaches per month can be associated one or more of the following symptoms: unilateral location, pulsating quality, moderate or severe pain intensity, aggravation by or causing avoidance of routine physical activity, nausea, vomiting, photophobia and phonophobia.

Globally, more than 10% of the population is affected by migraine at some point in life. Migraines most commonly start between 15 and 24 years of age and occur most frequently in those 35 to 45 years of age. In children, about 1.7% of 7 year olds and 3.9% of those between 7 and 15 years have migraines, with the condition being slightly more common in boys before puberty. During adolescence migraines becomes more common among women and this persists for the rest of the lifespan, being two times more common among elderly females than males. During premenopause symptoms often get worse before decreasing in severity. While symptoms resolve in about two thirds of the elderly, in between 3 and 10% they persist.

### ACTH

In some embodiments, ACTH is a 39 amino acid peptide hormone secreted by the anterior pituitary gland. ACTH is secreted from the anterior pituitary in response to corticotropin-releasing hormone (CRH) that is secreted from the hypothalamus. The release of ACTH stimulates the adrenal cortex with subsequent increased production of glucocorticosteroids and/or cortisol from the adrenal cortex.

In some embodiments, ACTH is synthesized from a precursor polypeptide pre-pro-opiomelanocortin (pre-POMC). The removal of the signal peptide during translation produces a 267 amino acid polypeptide POMC. POMC undergoes a series of post-translational modifications to yield various polypeptide fragments including and not limited to ACTH, β-lipotropin, γ-lipotropin, α, β, γ-Melanocyte Stimulating Hormone (MSH) and β-endorphin. POMC, ACTH and β-lipotropin are also secreted from the pituitary gland in response to the hormone corticotropin-releasing hormone (CRH). In some embodiments, the first 13 amino acids of ACTH₁₋₃₉ are cleaved to form α-melanocyte-stimulating hormone (α-MSH). In some embodiments, methods of preventing or treating an individual diagnosed with migraine (e.g., chronic migraine) comprise administration of a γ-MSH peptide, or fragment, analog, complex or aggregate thereof, or any combination thereof, to an individual in need thereof.

In some embodiments, multiple hypothalamic, pituitary, and peripheral factors regulate stress-mediated or inflammation-induced POMC expression and/or ACTH secretion. Essential cellular functions maintaining metabolic and neuroendocrine control require a homeostatic, non-stressed pattern of ACTH and glucocorticoid secretion. ACTH secretion is characterized by both circadian periodicity and ultradian pulsatility that is generated by CRH release and is also influenced by peripheral corticosteroids. Thus, ACTH secretion peaks at about before 7 am and nadir adrenal steroid secretion occurs between about 11 pm and 3 am, with periodic secretory bursts occurring throughout the day. Serum cortisol levels also exhibit a similar pattern of circadian periodicity. These rhythms are further reinforced by visual cues and the light-dark cycle. In some instances, stress results in increased ACTH pulse amplitude. In some instances, migraine is associated with abnormality in the circadian periodicity and ultradian pulsatility of ACTH and/or cortisol levels in the body.

In some instances, ACTH regulates astrocyte response by binding to melanocortin receptors. In some instances, ACTH suppresses neuroinflammation associated with migraine by binding to melanocortin receptors. In some instances, ACTH regulates migration of inflammatory cells and maintains the integrity of the blood brain barrier.

### DEFINITIONS

The term “ACTH” refers to corticotropin, adrenocorticotropic hormone, adrenocorticotropin or the like. The term “ACTH” also includes, but is not limited to, any ACTH peptide or any ACTH preparation as described herein. In some embodiments, ACTH is an ACTH peptide. As used herein, in some embodiments, “ACTH peptide” refers to ACTH₁₋₃₉ peptide of structure (e.g., having the sequence of the following):

(SEQ ID NO: 1)
H-Ser-Tyr-Ser-Met-Glu-His-Phe-Arg-Trp-Gly-
1 2 3 4 5 6 7 8 9 10
Lys-Pro-Val-Gly-Lys-Lys-Arg-Arg-Pro-Val-
11 12 13 14 15 16 17 18 19 20
Lys-Val-Tyr-Pro-Asp-Gly-Ala-Glu-Asp-Gln-
21 22 23 24 25 26 27 28 29 30
Leu-Ala-Glu-Ala-Phe-Pro-Leu-Glu-Phe-OH
31 32 33 34 35 36 37 38 39

or any homologs, analogs, fragments, complexes or aggregates thereof. The term ACTH includes peptides or peptide fragments, complexes, salts or aggregates with about 40%, about 45%, about 50%, about 55%, about 60%, about 65%, about 70%, about 75%, about 80%, about 85%, about 90%, or about 95% homology with ACTH₁₋₃₉ or with the corresponding fragment of ACTH in the case of fragments, and thus include the various ACTH peptides derived from mammalian sources. The term ACTH includes ACTH from any source including human ACTH, mouse ACTH, rat ACTH, porcine ACTH, sheep ACTH, bovine ACTH, rabbit ACTH or any other source of ACTH.

In some embodiments, the ACTH peptide or fragment, analog, complex, or aggregate thereof, or any combination thereof, is a porcine ACTH peptide or fragment, analog, complex, or aggregate thereof a human ACTH peptide or fragment, analog, complex, or aggregate thereof or a recombinant ACTH peptide or fragment, analog, complex, or aggregate thereof. In some embodiments, the ACTH peptide is a porcine ACTH peptide, a human ACTH peptide, or a recombinant ACTH peptide. In some embodiments, the ACTH peptide is a porcine ACTH peptide. In some embodiments, the ACTH peptide is a human ACTH peptide. In some embodiments, the ACTH peptide is a recombinant ACTH peptide. In some embodiments, the ACTH peptide is a recombinant human ACTH peptide.

In some embodiments, ACTH is an ACTH preparation. As used herein, “ACTH preparation” refers to a mixture containing ACTH peptide and/or other peptide fragments and/or other proteins and/or other substances that together form a composition that is suitable for any methods and/or dosing regimen described herein. In some of such embodiments, ACTH is obtained from a homogenized pituitary extract of an appropriate animal (e.g., pituitary extract of a pig). Any suitable method is used to obtain a homogenized pituitary extract. In some of such embodiments, a homogenized pituitary extract includes ACTH peptide and/or other peptide fragments and/or other proteins and/or other substances that are contemplated as being part of the ACTH preparation that is compatible with any method described herein.

The term ACTH includes humanized and/or recombinant forms of ACTH and synthetic forms of ACTH. The term ACTH includes fragments of ACTH₁₋₃₉. Examples of synthetic forms and/or fragments of ACTH include and are not limited to ACTH₁₋₂₄ peptide having the formula (e.g., having the sequence of the following) and also known as tetracosactide or Synacthen®:

(SEQ ID NO: 2)
H-Ser-Tyr-Ser-Met-Glu-His-Phe-Arg-Trp-Gly-
1 2 3 4 5 6 7 8 9 10
Lys-Pro-Val-Gly-Lys-Lys-Arg-Arg-Pro-Val-
11 12 13 14 15 16 17 18 19 20
Lys-Val-Tyr-Pro
21 22 23 24

or a fragment, complex, aggregate, or analog thereof, or any combination thereof,

ACTH₁₋₁₇ peptide having the formula (e.g., having the sequence of the following):

(SEQ ID NO: 3)
H-Ser-Tyr-Ser-Met-Glu-His-Phe-Arg-Trp-Gly-
1 2 3 4 5 6 7 8 9 10
Lys-Pro-Val-Gly-Lys-Lys-Arg-
11 12 13 14 15 16 17

or a fragment, complex, aggregate, or analog thereof, or any combination thereof,

ACTH₄₋₁₀ peptide (ORG-066) of formula (e.g., having the sequence of the following):

(SEQ ID NO: 4)
Met-Glu-His-Phe-Arg-Trp-Gly
4 5 6 7 8 9 10

or a fragment, complex, aggregate, or analog thereof, or any combination thereof, or

ACTH₄₋₉ peptide analog (ORG-2766, N-[2-Amino-4-(methylsulfonyl)butanoyl]-L-α-glutamyl-L-histidyl-L-phenylalanyl-D-lysyl-L-phenylalanine) of formula (e.g., having the sequence of the following):

(SEQ ID NO: 5)
O₂-Met-Glu-His-Phe-D-Lys-Phe-OH

or a fragment, complex, aggregate, or analog thereof, or any combination thereof.

The term ACTH includes a peptide of formula (e.g., having the sequence of the following):

(SEQ ID NO: 6)
Ac-Ser-Tyr-Ser-Met-Glu-His-Phe-Arg-Trp-Gly-Lys-
Pro-Val-NH₂

or a fragment, complex, aggregate, or analog thereof, or any combination thereof, or a peptide fragment of formula (e.g., having the sequence of the following):

(SEQ ID NO: 7)
H-Ser-Tyr-Ser-Met-Glu-His-Phe-Arg-Trp-Gly-Lys-Pro-
Val

or a fragment, complex, aggregate, or analog thereof, or any combination thereof, or a peptide fragment of formula (e.g., having the sequence of the following):

(SEQ ID NO: 8)
D-Ala-Gln-Tyr-Phe-Arg-Trp-Gly-NH₂

or a fragment, complex, aggregate, or analog thereof, or any combination thereof. As illustrated by the above examples the ACTH peptide, fragment, complex, aggregate of analog thereof may also have C- and N-terminal modifications, such as acetylation (CH₃C(O)— at the N-terminus and amidation (—NH₂ or substituted —N in place of —OH) at the C-terminus to provide a carboxamide, such modifications may be present naturally from post translational modification in vivo or may be introduced synthetically.

The term ACTH also includes synthetic and natural extract preparations of ACTH and fragments that are commercially available including and not limited to ACTHAR® powder for injection or gel, Synacthen®, Adrenomone®, or the like. ACTHAR® is derived from porcine pituitary glands and the ACTH obtained therefrom has the sequence of SEQ ID NO: 1. Examples of commercially available ACTH peptides that are compatible with the methods described herein include and are not limited to Adrenocorticotropic Hormone (ACTH) (1-10) (human), Adrenocorticotropic Hormone (ACTH) (1-13) (human), Adrenocorticotropic Hormone (ACTH) (1-16) (human), Adrenocorticotropic Hormone (ACTH) (1-17) (human), Adrenocorticotropic Hormone (ACTH) (1-24) (human) (Synacthen®), Adrenocorticotropic Hormone (ACTH) (1-39) (human), Adrenocorticotropic Hormone (ACTH) (1-39) (porcine), Adrenocorticotropic Hormone (ACTH) (1-39) (rat), Adrenocorticotropic Hormone (ACTH) (18-39) (human), Adrenocorticotropic Hormone (ACTH) (4-10) (human), Adrenocorticotropic Hormone (ACTH) (1-4), Adrenocorticotropic Hormone (ACTH) (1-14) or the like available from, for example, GenScript.

As used herein, the term ACTH also includes pre-POMC, POMC, β-lipotropin, γ-lipotropin, Melanocyte Stimulating Hormone (α-MSH, β-MSH, γ-MSH), β-endorphin, or the like, or any other polypeptide fragment that is a post-translational product of the POMC gene. POMC genes for various species are found in the NCBI GenBank including and not limited to human POMC transcript variant 1, mRNA, (NCBI Accession number NM_{—}001035256), human POMC transcript variant 2, mRNA, (NCBI Accession number NM_{—}000939), swine pro-opiomelanocortin, mRNA (NCI Accession number S73519), swine proopiomelanocortin protein (POMC) gene (NCBI Accession number EU184858), rat proopiomelanocortin (POMC) gene (NCBI Accession number K01877), or the like. Other examples of POMC genes include, for example, catfish POMC gene described in Animal Genetics, 2005, 36, 160-190.

The term “ACTH analog” or “analog of ACTH” refers to any compounds in which one or more atoms, functional groups, or substructures or amino acids in ACTH or fragments of ACTH have been replaced with different atoms, groups, or substructures or amino acids while retaining the functional activity of the ACTH or fragments of ACTH. In some embodiments, an ACTH analog is a peptide segment of ACTH₁₋₃₉ peptide that retains biological activity of ACTH.

The term “ACTH complex” refers to ACTH or fragments or analogs thereof that are optionally complexed with other proteins (e.g., Bovine Serum Albumin) or metal ions, or fragments of ACTH, or any other suitable complexes that retain the functional characteristics of ACTH or ACTH fragments or analogs thereof and/or allow for formulation of ACTH or ACTH fragments or analogs thereof into suitable dosage forms.

The term “acute” referring to a condition is one with a rapid onset and/or a short course and that could remit for periods of time and then recur, such as for example migraines that occur somewhat infrequently, or are serious conditions such as status migrainosus, that require a short course of therapy to speed up their resolution, and “acute administration” involves dosing as soon as possible to the onset of symptoms or aura, if present, to foreclose a full-blown migraine episode.

The term “chronic” referring to a condition is one that is persistent or otherwise long-lasting in its effects, a non-limiting example being chronic migraine and the term “chronic administration” refers to dosing meant to treat the persistent or recurring condition, typically by extended periods of dosing.

The terms “preventing” or “prevent” or “prophylaxis” or “prophylactic” when referring to methods of treatment herein described refers to the halting or lessening of the frequency and/or severity of the condition being described, and such treatment involves the chronic (on-going) administration of a treatment agent

The term “prodrug” refers to a precursor molecule that is a derivative of ACTH or ACTH fragments or analogs thereof that is suitable for incorporation in any dosage form described herein. A “prodrug” refers to a precursor compound that is converted into active compound in vivo. Prodrugs are often useful because, in some situations, they may be easier to administer than the parent drug. They may, for instance, be bioavailable by oral administration whereas the parent is not. The prodrug may also have improved solubility in pharmaceutical compositions over the parent drug. In some embodiments, prodrugs facilitate transmittal across a cell membrane where water solubility is detrimental to mobility but which then is metabolically hydrolyzed to a polar group, the active entity, once inside the cell where water-solubility is beneficial. As non-limiting examples, a prodrug of ACTH or fragment of analog thereof is metabolically stable and is not degraded in the stomach, but capable of conversion to the active entity in the blood stream or in a cell.

Prodrugs are generally drug precursors that, following administration to a subject and subsequent absorption, are converted to an active, or a more active species via some process, such as conversion by a metabolic pathway. Some prodrugs have a chemical group present on the prodrug that renders it less active and/or less labile and/or confers solubility or some other property to the drug. Once the chemical group has been cleaved and/or modified from the prodrug the active drug is generated. In some embodiments, a prodrug of ACTH or fragment or analog thereof is an alkyl ester of the parent compound such as, for example, methyl ester, ethyl ester, n-propyl ester, iso-propyl ester, n-butyl ester, sec-butyl ester, tert-butyl ester or any other pharmaceutically acceptable ester.

A “therapeutically effective amount” means the amount that, when administered to an animal for treating a disease, is sufficient to effect treatment for that disease.

“Treating” or “treatment” of a disease includes preventing the disease from occurring in an individual that may be predisposed to the disease but does not yet experience or exhibit symptoms of a migraine episode (prophylactic treatment), inhibiting the disease such as in acute treatment during migraine aura (slowing or arresting its development), providing acute relief from the symptoms or side-effects of a migraine episode (including palliative treatment, such as for relieving status migrainosus).

### Methods

Provided herein are methods of treating, including preventing, migraine headache (e.g., chronic migraine) comprising administration of adrenocorticotropic hormone (ACTH) peptide or fragment, analog, complex or aggregate thereof, or any combination thereof, as described herein, to individuals in need thereof. In some embodiments, methods of treatment of migraine headache described herein allow for early intervention upon detection of adverse effects such as fluctuating blood glucose levels, weight gain, irritability, fluctuating blood pressure, edema, rash, allergic reaction, Cushinoid Syndrome, or any other intolerable symptoms.

In some embodiments, methods of preventing or treating chronic migraine in an individual in need thereof comprise selecting an individual diagnosed with chronic migraine and administering an adrenocorticotropic hormone (ACTH) peptide, or fragment, analog, complex or aggregate thereof, or any combination thereof, to the individual in need thereof.

In some embodiments, the methods of alleviating the symptoms of migraine headache described herein, prevents or down-regulates CGRP release from trigeminal nerve endings.

In some embodiments the methods of alleviating the symptoms of migraine headache described herein upregulate plasma cortisol, which reduces CRH output through a negative feedback loop, which in turn prevents mast cell degranulation which may occur in propagation and maintenance of chronic migraine headache.

In some embodiments, the methods described herein may be used for the treatment of an individual diagnosed with migraine wherein the individual's migraine is refractory to other migraine treatments. In some embodiments, the methods described herein may be used for the treatment of an individual diagnosed with migraine wherein the individual's migraine is refractory to onabotulinum toxin A (Botox®) treatment. In some embodiments, the methods described herein may be used for the treatment of an individual diagnosed with migraine wherein the individual is contraindicated for treatment with another migraine medication. In further embodiments, the contraindicated treatment is chosen from triptans, and an ergot derivative, including dihydroergotamine (DHE, Migranal®, Levadex®), methysergide, methylergonovine and methylergometrine.

### Dosing Regimen

In some embodiments, the ACTH peptide or fragment, analog, complex or aggregate thereof, or combination thereof is administered in an initial phase of from one to about five doses at daily intervals and a subsequent phase of one or more doses at intervals greater than one day. In some embodiments the interval for administration of the second phase doses is every other day, every two days, every three days, every four days, every 5 days, every 6 days, once a week, or every two weeks. In some embodiments, the ACTH peptide or fragment, analog, complex or aggregate thereof, or any combination thereof, may be administered as a first dose, followed by one or more subsequent doses. In some embodiments of the methods of treatment of migraine headache described herein, the first dose and one or more subsequent doses of ACTH or fragment, analog, complex or aggregate thereof, or any combination thereof, are administered in a dosing regimen that is not continuous (e.g., the intervals between doses are uneven). In some embodiments of the methods of treatment of migraine headache described above, the first dose and one or more subsequent doses of ACTH or fragment, analog, complex or aggregate thereof, or any combination thereof, are administered in a dosing regimen that is a continuous dosing regimen. In some embodiments, the ACTH peptide or fragment, analog, complex or aggregate thereof, or any combination thereof, may be administered as a first dose, followed by one or more subsequent doses, wherein the subsequent dose values are equivalent to the initial dose value. In some embodiments, the ACTH peptide or fragment, analog, complex or aggregate thereof, or any combination thereof, may be administered as a first dose, followed by one or more subsequent doses, wherein the subsequent dose values are not equivalent to the initial dose value. In some embodiments, the first dose is administered upon detection of one or more symptoms of migraine headache in a clinically diagnosed migraine headache patient. In some embodiments, the one or more subsequent doses are administered every day, every other day, every two days, every three days, every four days, every 5 days, every 6 days, once a week, every two weeks, every three weeks, once a month, every six weeks, every two months, every three months, every four months five months, every six months or any combination thereof. In some embodiments, the ACTH peptide or fragment, analog, complex or aggregate thereof is administered acutely at onset of migraine, or of aura for migraine with aura, or for an episode of status migrainosus and administration is continued every day, about every 2 days, about every 5 days, about every week, about every two weeks, about every three weeks, about every month, about every two months, or any combination thereof as needed to sustain the therapeutic effect until the condition remits. In some embodiments, the ACTH peptide or fragment, analog, complex or aggregate thereof is administered about every day, about every 2 days, about every 5 days, about every week, about every two weeks, about every three weeks, about every month, about every two months, or any combination thereof before onset of migraine and for as long as the patient experiences migraine symptoms in the absence of said ACTH peptide or fragment, analog, complex or aggregate thereof.

In some embodiments, the dosing regimen comprises doses that produce decreasing levels of drug early in the dosing interval followed by a prolonged dose-free interval. In some embodiments, the dosing regimen comprises a first dose, a series of subsequent doses, followed by a drug holiday, and then, one or more series of doses that are the same as or different from the first series of doses. By way of example only, in one dosing regimen, the methods of treatment of migraine headache described above comprise administration of ACTH or fragment, analog, complex or aggregate thereof, or any combination thereof, and comprise a first dose of 80 IU, then a once daily dose of 20 IU for three days, followed by a 40 IU dose every week for a month, followed by a period of no treatment for 3 months, and then a second series of doses comprising a first dose of 60 IU, then a once daily dose of 20 IU for three days, followed by a 40 IU dose every week for a month, followed by a period of no treatment for 3 months.

In some embodiments, a dosing regimen comprises dosing that produces escalating levels of drug early in the dosing interval followed by a prolonged dose-free period. By way of example only, in one dosing regimen, the methods of treatment of migraine headache describe above comprise administration of ACTH or fragment, analog, complex or aggregate thereof, or any combination thereof, and comprise a first dose of 20 IU, a second dose of 20 IU in the same week, then 40 IU twice a week, then 40 IU every other month for three months.

In some embodiments, a first dose of ACTH or fragment, analog, complex or aggregate thereof, or any combination thereof, is between about 10 IU, 20 IU, 30 IU, 40 IU, 50 IU, 60 IU, 70 IU, 80 IU and about 50 IU, 60 IU, 70 IU, 80 IU, 90 IU, 100 IU, 110 IU, 120 IU, 130 IU, 140 IU, 150 IU or 200 IU. In some embodiments, a first dose of ACTH or fragment, analog, complex or aggregate thereof, or any combination thereof, is between about 10 IU to about 200 IU, between about 10 IU to about 150 IU, between about 10 IU to about 100 IU, between about 10 IU to about 80 IU, between about 10 IU to about 60 IU, or between about 10 IU to about 40 IU. In some embodiments, a first dose of ACTH or fragment, analog, complex or aggregate thereof, or any combination thereof, is between about 10 IU to about 200 IU, between about 20 IU to about 200 IU, between about 40 IU to about 200 IU, between about 40 IU to about 150 IU, between about 40 IU to about 100 IU, between about 40 IU to about 80 IU, or between about 40 IU to about 60 IU. In some embodiments, a first dose of ACTH or fragment, analog, complex or aggregate thereof, or any combination thereof, is between about 20 IU to about 200 IU, between about 60 IU to about 150 IU, between about 60 IU to about 100 IU, or between about 60 IU to about 80 IU.

In some embodiments, one or more subsequent dose of ACTH or fragment, analog, complex or aggregate thereof, or any combination thereof, is between about 10 IU, 20 IU, 30 IU, 40 IU, 50 IU, 60 IU, 70 IU, 80 IU to about 50 IU, 60 IU, 70 IU, 80 IU, 90 IU, 100 IU, 110 IU, 120 IU, 130 IU, 140 IU, 150 IU or 200 IU. In some embodiments, a one or more subsequent dose of ACTH or fragment, analog, complex or aggregate thereof, or any combination thereof, is between about 10 IU to about 200 IU, between about 10 IU to about 150 IU, between about 10 IU to about 100 IU, between about 10 IU to about 80 IU, between about 10 IU to about 60 IU, or between about 10 IU to about 40 IU. In some embodiments, a one or more subsequent dose of ACTH or fragment, analog, complex or aggregate thereof, or any combination thereof, is between about 20 IU to about 200 IU, between about 20 IU to about 150 IU, between about 20 IU to about 100 IU, between about 20 IU to about 80 IU, or between about 20 IU to about 60 IU, or between about 20 IU to about 40 IU. In some embodiments, a one or more subsequent dose of ACTH or fragment, analog, complex or aggregate thereof, or any combination thereof, is between about 40 IU to about 200 IU, between about 40 IU to about 150 IU, between about 40 IU to about 100 IU, between about 40 IU to about 80 IU, or between about 40 IU to about 60 IU. In some embodiments, a one or more subsequent dose of ACTH or fragment, analog, complex or aggregate thereof, or any combination thereof, is between about 20 IU to about 200 IU, between about 60 IU to about 150 IU, between about 60 IU to about 100 IU, or between about 60 IU to about 80 IU.

In some embodiments, the ACTH, or fragment, analog, complex or aggregate thereof, or any combination thereof, is administered as a synthetic preparation (i.e., not naturally occurring), wherein a first dose of ACTH or fragment, analog, complex or aggregate thereof, or any combination thereof, is between about 10 mg/kg, 20 mg/kg, 30 mg/kg, 40 mg/kg, 50 mg/kg, 60 mg/kg, 70 mg/kg, 80 mg/kg to about 50 mg/kg, 60 mg/kg, 70 mg/kg, 80 mg/kg, 90 mg/kg, 100 mg/kg, 110 mg/kg, 120 mg/kg, 130 mg/kg, 140 mg/kg, 150 mg/kg or 200 mg/kg. In some embodiments, a first dose of ACTH or fragment, analog, complex or aggregate thereof, or any combination thereof, is between about 10 mg/kg to about 200 mg/kg, between about 20 mg/kg to about 200 mg/kg, between about 20 mg/kg to about 150 mg/kg, between about 20 mg/kg to about 100 mg/kg, between about 20 mg/kg to about mg/kg IU, between about mg/kg IU to about mg/kg IU, or between about 20 mg/kg to about 40 mg/kg. In some embodiments, a first dose of ACTH or fragment, analog, complex or aggregate thereof, or any combination thereof, is between about 40 mg/kg to about 200 mg/kg, between about 40 mg/kg to about 150 v, between about 40 mg/kg to about 100 mg/kg, between about 40 mg/kg to about 80 mg/kg, or between about 40 mg/kg to about 60 mg/kg. In some embodiments, a first dose of ACTH or fragment, analog, complex or aggregate thereof, or any combination thereof, is between about 20 mg/kg to about 200 mg/kg, between about 60 mg/kg to about 150 mg/kg, between about 60 mg/kg to about 100 mg/kg, or between about 60 mg/kg to about 80 mg/kg.

Where the ACTH, or fragment, analog, complex or aggregate thereof, or any combination thereof, is a synthetic preparation (i.e., not naturally occurring), in some embodiments, one or more subsequent dose of ACTH or fragment, analog, complex or aggregate thereof, or any combination thereof, is between about 10 mg/kg, 20 mg/kg, 30 mg/kg, 40 mg/kg, 50 mg/kg, 60 mg/kg, 70 mg/kg, 80 mg/kg to about 50 mg/kg, 60 mg/kg, 70 mg/kg, 80 mg/kg, 90 mg/kg, 100 mg/kg, 110 mg/kg, 120 mg/kg, 130 mg/kg, 140 mg/kg, 150 mg/kg or 200 mg/kg. In some embodiments, one or more subsequent dose of ACTH or fragment, analog, complex or aggregate thereof, or any combination thereof, is between about 10 mg/kg to about 200 mg/kg, between about 10 mg/kg to about 150 mg/kg, between about 10 mg/kg to about 100 mg/kg, between about 10 mg/kg to about 80 mg/kg IU, between about 10 mg/kg IU to about 60 mg/kg IU, or between about 10 mg/kg to about 40 mg/kg. In some embodiments, one or more subsequent dose of ACTH or fragment, analog, complex or aggregate thereof, or any combination thereof, is between about 20 mg/kg to about 200 mg/kg, between about 20 mg/kg to about 150 mg/kg, between about 20 mg/kg to about 100 mg/kg, between about 20 mg/kg to about 80 mg/kg, or between about 20 mg/kg to about 60 mg/kg. In some embodiments, a one or more subsequent dose of ACTH or fragment, analog, complex or aggregate thereof, or any combination thereof, is between about 40 mg/kg to about 200 mg/kg, between about 40 mg/kg to about 150 mg/kg, between about 40 mg/kg to about 100 mg/kg, between about 40 mg/kg to about 80 mg/kg, or between about 40 mg/kg to about 60 mg/kg. In some embodiments a one or more subsequent dose of ACTH or fragment, analog, complex or aggregate thereof, or any combination thereof, is between about 20 mg/kg to about 200 mg/kg, between about 60 mg/kg to about 150 mg/kg, between about 60 mg/kg to about 100 mg/kg, or between about 60 mg/kg to about 80 mg/kg.

In some embodiments, a first dose of ACTH or fragment, analog, complex or aggregate thereof, or any combination thereof, is between about 0.01 mg to about 10 mg, between about 0.1 mg to about 2.5 mg, or between about 2 mg to about 5 mg. In some embodiments, a first dose of ACTH or fragment, analog, complex or aggregate thereof, or any combination thereof is about 0.25 mg, about 0.5 mg, about 0.75 mg, about 1.0 mg, about 1.25 mg, about 1.5 mg, about 1.75 mg, about 2.0 mg, about 2.25 mg, about 2.5 mg, about 2.75 mg, about 3.0 mg, about 3.25 mg, about 3.5 mg, about 3.75 mg, about 4.0 mg, about 4.25 mg, about 4.5 mg, about 4.75 mg, or about 5 mg.

In some embodiments, one or more subsequent dose of ACTH or fragment, analog, complex or aggregate thereof, or any combination thereof, is between about 0.01 mg to about 10 mg, between about 0.1 mg to about 2.5 mg, or between about 2 mg to about 5 mg. In some embodiments, one or more subsequent dose of ACTH or fragment, analog, complex or aggregate thereof, or any combination thereof is about 0.25 mg, about 0.5 mg, about 0.75 mg, about 1.0 mg, about 1.25 mg, about 1.5 mg, about 1.75 mg, about 2.0 mg, about 2.25 mg, about 2.5 mg, about 2.75 mg, about 3.0 mg, about 3.25 mg, about 3.5 mg, about 3.75 mg, about 4.0 mg, about 4.25 mg, about 4.5 mg, about 4.75 mg, or about 5 mg.

In some embodiments, one or more subsequent dose of ACTH or fragment, analog, complex or aggregate thereof, or any combination thereof, is between about 10% and about 90%, between about 20% and about 80%, between about 20% and about 60%, or between about 20% -and about 40% of the first dose of ACTH or fragment, analog, complex or aggregate thereof, or any combination thereof. In some embodiments, one or more subsequent dose of ACTH or fragment, analog, complex or aggregate thereof, or any combination thereof, is between about 80% and about 200%, between about 80% and about 175%, between about 80% and about 150%, between about 80% and about 125%, between about 80% and about 100%, between about 100% and 200%, between about 100% and 150%, or between about 100% and 120% of the first dose of ACTH or fragment, analog, complex or aggregate thereof, or any combination thereof. In some embodiments, one or more subsequent dose of ACTH or fragment, analog, complex or aggregate thereof, or any combination thereof, is 100% of the first dose of ACTH or fragment, analog, complex or aggregate thereof, or any combination thereof.

In some embodiments, the ACTH peptide or fragment, analog, complex or aggregate thereof, or any combination thereof, is administered to an individual in need thereof in an amount sufficient to provide plasma cortisol secretion levels between about 1.1 to about 10 times 1.1 to about 8 times, 1.1 to about 6 times, 1.1 to about 4 times, between about 1.1 to about 3 times, between about 1.1 to about 2 times, between about 1.1 to about 1.5 times the plasma cortisol secretion levels of a normal individual at about 8 am. In some embodiments, the ACTH peptide or fragment, analog, complex or aggregate thereof, or any combination thereof, is administered to an individual in need thereof in an amount sufficient to provide plasma cortisol secretion levels between about 1.5 to about 4 times, between about 1.5 to about 3 times, or between about 1.15 to about 2 times, the plasma cortisol secretion levels of a normal individual at about 8 am.

In some embodiments, the ACTH peptide or fragment, analog, complex or aggregate thereof, or any combination thereof, is administered to an individual in need thereof in an amount sufficient to provide plasma cortisol secretion levels between about 1.1 to about 10 times 1.1 to about 8 times, 1.1 to about 6 times, 1.1 to about 4 times, between about 1.1 to about 3 times, between about 1.1 to about 2 times, or between about 1.1 to about 1.5 times the plasma cortisol secretion levels prior to administration of the ACTH peptide or fragment, analog, complex or aggregate thereof, or any combination thereof. In some embodiments, the ACTH peptide or fragment, analog, complex or aggregate thereof, or any combination thereof, is administered to an individual in need thereof in an amount sufficient to provide plasma cortisol secretion levels between about 1.5 to about 4 times, between about 1.5 to about 3 times, or between about 1.15 to about 2 times, the plasma cortisol secretion levels prior to administration of the ACTH peptide or fragment, analog, complex or aggregate thereof, or any combination thereof.

In some embodiments, the ACTH peptide or fragment, analog, complex or aggregate thereof, or any combination thereof, is administered in an amount sufficient to provide plasma cortisol concentration between about 1.5 to about 120 μg/100 mL over at least 24 hours after administration. In some embodiments, the ACTH peptide or fragment, analog, complex or aggregate thereof, or any combination thereof, is administered in an amount sufficient to provide plasma cortisol concentration between about 1.5 to about 60 μg/100 mL over at least 24 hours after administration. In some embodiments, the ACTH peptide or fragment, analog, complex or aggregate thereof, or any combination thereof, is administered in an amount sufficient to provide plasma cortisol concentration between about 1.5 to about 30 μg/100 mL over at least 24 hours after administration.

In some embodiments, where the patient's condition does not improve upon administration of a dosing regimen described herein, upon the doctor's discretion the ACTH peptide or fragment, analog, complex or aggregate thereof, or any combination thereof is optionally administered chronically, that is, for an extended period of time, including throughout the duration of the patient's life in order to ameliorate or otherwise control or limit the symptoms of the patient's disease or condition.

In the case wherein the patient's status does improve, upon the doctor's discretion the administration of the ACTH peptide or fragment, analog, complex or aggregate thereof, or any combination thereof is optionally given continuously; alternatively, the dose of ACTH peptide or fragment, analog, complex or aggregate thereof, or any combination thereof being administered is temporarily reduced or temporarily suspended for a certain length of time (i.e., a “drug holiday”). The length of the drug holiday optionally varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday includes from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, the dosage or the frequency of administration, or both, is reduced, as a function of the symptoms, to a level at which the improved disease, disorder or condition is retained. In some embodiments, patients require intermittent treatment on a long-term basis upon any recurrence of symptoms.

In some embodiments, the pharmaceutical compositions described herein are in unit dosage forms suitable for single administration of precise dosages. In unit dosage form, the formulation is divided into unit doses containing appropriate quantities of ACTH peptide or fragment, analog, complex or aggregate thereof, or any combination thereof. In some embodiments, the unit dosage is in the form of a package containing discrete quantities of the formulation. Non-limiting examples are packaged tablets or capsules, powders in vials or ampoules, or injectable suspension or solution in ampoules. In some embodiments, aqueous suspension compositions are packaged in single-dose non-reclosable containers. Alternatively, multiple-dose reclosable containers are used. In some of such embodiments, a preservative is optionally included in the composition. By way of example only, formulations for intramuscular injection are presented in unit dosage form, which include, but are not limited to ampoules, or in multi dose containers, with an added preservative.

ACTH(1-39) and ACTH(1-24) dosing is known in humans as these peptides have been used in human therapy and diagnostics for other conditions. The dosage regimens described herein are advantageous for the methods of treatment described herein. Doses of fragments and analogs may be dosed based on experimental pharmacokinetic measurements of plasma half-lives and efficacy at the melanocortin receptors.

### Combination Therapy

In some embodiments of the methods and dosing regimens described above, ACTH peptide or fragment, analog, complex or aggregate thereof, or any combination thereof, is administered in combination with other agents including, and not limited to: antiepileptics, including topiramate gabapentin, pregabalin, valproate sodium, and zonisamide; onabotulinumtoxin A (Botox®, Dysport®) and botulinum toxin B (Neurobloc®, Myobloc®), tizanidine, tricyclic antidepressants (TCA's), including amitriptyline, imipramine, nortriptyline, protriptyline and doxepin; fluoxetine; memantine; triptans, including sumatriptan (Imitrex®, Treximet®), frovatriptan (Frova®), almotriptan (Axert®), rizatriptan (Maxalt®), zolmitriptan (Zomig®); dihydroergotamine (DHE, Migranal®, Levadex®), metoclopramide, hydroxyzine; ergot derivatives, including methysergide, methylergonovine and methylergometrine; neuroleptics, including prochlorperazine, chlorpromazine and droperidol; corticosteroids; magnesium; NSAIDS, including ketorolac, naproxen sodium and acetaminophen; opioids, antiemetics (metoclopramide, ondansetron), vitamin B12, timolol or propranolol.

In some embodiments of combination therapy, the ACTH peptide or fragment, analog, complex or aggregate thereof, or any combination thereof, and the second therapeutic agent are administered simultaneously. In some embodiments of combination therapy, the ACTH peptide or fragment, analog, complex or aggregate thereof, or any combination thereof, and the second therapeutic agent are administered serially in any order. In some embodiments of combination therapy, the ACTH peptide or fragment, analog, complex or aggregate thereof, or any combination thereof, and the second therapeutic agent are administered at different intervals. By way of example only, a second therapeutic agent is administered after completion of a dosing regimen comprising administration of ACTH peptide or fragment, analog, complex or aggregate thereof, or any combination thereof, or is administered adjunctively if breakthrough migraine occurs despite prophylactic ACTH dosing, such as by administration of a triptan or dihydroergotamine.

### Pharmaceutical Formulations

Provided herein, in certain embodiments, are compositions comprising at least one ACTH peptide or fragment, analog, complex or aggregate thereof, or any combination thereof, where the ACTH peptide or fragment, analog, complex or aggregate thereof, or any combination thereof, is as described herein.

Pharmaceutical compositions are formulated using one or more physiologically acceptable carriers including excipients and auxiliaries which facilitate processing of the ACTH peptide or fragment, analog, complex or aggregate thereof, or any combination thereof, into preparations which are used pharmaceutically. Proper formulation is dependent upon the route of administration chosen. A summary of pharmaceutical compositions is found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins, 1999).

Provided herein are pharmaceutical compositions that include one or more of ACTH peptide or fragment, analog, complex or aggregate thereof, or any combination thereof, and a pharmaceutically acceptable diluent(s), excipient(s), or carrier(s). In addition, the ACTH peptide or fragment, analog, complex or aggregate thereof, or any combination thereof, is optionally administered as pharmaceutical compositions in which it is mixed with other active ingredients, as in combination therapy. In some embodiments, the pharmaceutical compositions includes other medicinal or pharmaceutical agents, carriers, adjuvants, such as preserving, stabilizing, wetting or emulsifying agents, solution promoters, salts for regulating the osmotic pressure, and/or buffers. In addition, the pharmaceutical compositions also contain other therapeutically valuable substances.

A pharmaceutical composition, as used herein, refers to a mixture of ACTH peptide or fragment, analog, complex or aggregate thereof, or any combination thereof, with other chemical components, such as carriers, stabilizers, diluents, dispersing agents, suspending agents, thickening agents, and/or excipients. In some embodiments, a pharmaceutical composition comprises an ACTH preparation (e.g., an ACTH peptide or fragment, analog, complex or aggregate thereof, or any combination thereof, and any other proteins and/or other substances that are present in a homogenized pituitary extract obtained from an appropriate animal source) and other chemical components, such as carriers, stabilizers, diluents, dispersing agents, suspending agents, thickening agents, and/or excipients. The pharmaceutical composition facilitates administration of ACTH peptide or fragment, analog, complex or aggregate thereof, or any combination thereof, to an organism. In practicing the methods of treatment or use provided herein, an ACTH peptide or fragment, analog, complex or aggregate thereof, or any combination thereof, are administered in a pharmaceutical composition to a mammal having a condition, disease, or disorder to be treated. Preferably, the mammal is a human. The dose and dosing regimen varies depending on the severity and stage of the condition, the age and relative health of an individual, the potency of ACTH peptide or fragment, analog, complex or aggregate thereof, or any combination thereof, use and other factors. The ACTH peptide or fragment, analog, complex or aggregate thereof, or any combination thereof, is optionally used singly or in combination with one or more therapeutic agents as components of mixtures.

The pharmaceutical formulations described herein are optionally administered to a individual by multiple administration routes, including but not limited to, oral, parenteral (e.g., intravenous, subcutaneous, intramuscular, intrathecal), intranasal, buccal, topical, rectal, or transdermal administration routes. The pharmaceutical formulations described herein include, but are not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations, and mixed immediate and controlled release formulations.

The pharmaceutical compositions will include at least one ACTH peptide or fragment, analog, complex or aggregate thereof, or any combination thereof, as an active ingredient in free-acid or free-base form, or in the form of a pharmaceutically acceptable salt, ester or amide. In addition, the methods and pharmaceutical compositions described herein include the use of N-oxides, crystalline forms (also known as polymorphs), as well as active metabolites of ACTH peptide or fragment, analog, complex or aggregate thereof, or any combination thereof, having the same type of activity. In some situations, ACTH peptide or fragment, analog, complex or aggregate thereof, or any combination thereof, exist as tautomers and/or rotational isomers. All tautomers and/or rotational isomers are included within the scope of the embodiments presented herein. Additionally, ACTH peptide or fragment, analog, complex or aggregate thereof, or any combination thereof, exists in unsolvated as well as solvated forms with pharmaceutically acceptable solvents such as water, ethanol, and the like. The solvated forms of the ACTH peptide or fragment, analog, complex or aggregate thereof, or any combination thereof, presented herein are also considered to be disclosed herein. In some embodiments, ACTH peptide or fragment, analog, complex or aggregate thereof, or any combination thereof, exists as a complex with metal ions. The metal-ion complexed forms of the ACTH peptide or fragment, analog, complex or aggregate thereof, or any combination thereof, presented herein are also considered to be disclosed herein.

“Carrier materials” include any commonly used excipients in pharmaceutics and should be selected on the basis of compatibility with ACTH peptide or fragment, analog, complex or aggregate thereof, or any combination thereof, disclosed herein, and the release profile properties of the desired dosage form. Exemplary carrier materials include, e.g., binders, suspending agents, disintegration agents, filling agents, surfactants, solubilizers, stabilizers, lubricants, wetting agents, diluents, and the like.

Moreover, the pharmaceutical compositions described herein, which include an ACTH peptide or fragment, analog, complex or aggregate thereof, or any combination thereof, are formulated into any suitable dosage form, including but not limited to, aqueous oral dispersions, liquids, gels, syrups, elixirs, slurries, suspensions and the like, for oral ingestion by a patient to be treated, solid oral dosage forms, aerosols, controlled release formulations, fast melt formulations, effervescent formulations, lyophilized formulations, tablets, powders, pills, dragees, capsules, delayed release formulations, extended release formulations, pulsatile release formulations, multiparticulate formulations, and mixed immediate release and controlled release formulations. In some embodiments, a formulation comprising an ACTH peptide or fragment, analog, complex or aggregate thereof, or any combination thereof, is a solid drug dispersion. A solid dispersion is a dispersion of one or more active ingredients in an inert carrier or matrix at solid state prepared by the melting (or fusion), solvent, or melting-solvent methods. (Chiou and Riegelman, Journal of Pharmaceutical Sciences, 60, 1281 (1971)). The dispersion of one or more active agents in a solid diluent is achieved without mechanical mixing. Solid dispersions are also called solid-state dispersions. In some embodiments, any ACTH peptide or fragment, analog, complex or aggregate thereof, or any combination thereof, described is formulated as a spray dried dispersion (SDD). An SDD is a single phase amorphous molecular dispersion of a drug in a polymer matrix. It is a solid solution prepared by dissolving the drug and a polymer in a solvent (e.g., acetone, methanol or the like) and spray drying the solution. The solvent rapidly evaporates from droplets which rapidly solidifies the polymer and drug mixture trapping the drug in amorphous form as an amorphous molecular dispersion. In some embodiments, such amorphous dispersions are filled in capsules and/or constituted into powders for reconstitution. Solubility of an SDD comprising a drug is higher than the solubility of a crystalline form of a drug or a non-SDD amorphous form of a drug. In some embodiments of the methods described herein, ACTH peptide or fragment, analog, complex or aggregate thereof, or any combination thereof, are administered as SDDs constituted into appropriate dosage forms described herein.

Pharmaceutical preparations for oral use are optionally obtained by mixing one or more solid excipient with an ACTH peptide or fragment, analog, complex or aggregate thereof, or any combination thereof, optionally grinding the resulting mixture, and processing the mixture of granules, after adding suitable auxiliaries, if desired, to obtain tablets or dragee cores. Suitable excipients include, for example, fillers such as sugars, including lactose, sucrose, mannitol, or sorbitol; cellulose preparations such as, for example, maize starch, wheat starch, rice starch, potato starch, gelatin, gum tragacanth, methylcellulose, microcrystalline cellulose, hydroxypropylmethylcellulose, sodium carboxymethylcellulose; or others such as: polyvinylpyrrolidone (PVP or povidone) or calcium phosphate. If desired, disintegrating agents are added, such as the cross linked croscarmellose sodium, polyvinylpyrrolidone, agar, or alginic acid or a salt thereof such as sodium alginate. In some embodiments, a prodrug of the ACTH peptide or fragment, analog, complex or aggregate thereof, or any combination thereof, is used in preparations for oral use.

Dragee cores are provided with suitable coatings. For this purpose, concentrated sugar solutions are generally used, which optionally contain gum arabic, talc, polyvinylpyrrolidone, carbopol gel, polyethylene glycol, and/or titanium dioxide, lacquer solutions, and suitable organic solvents or solvent mixtures. Dyestuffs or pigments are optionally added to the tablets or dragee coatings for identification or to characterize different combinations of active compound doses.

In some embodiments, the liquid dosage forms disclosed herein are packaged in a multidose device that can be set to administer a precise dosage of the active ingredient, via, for example, subcutaneous injection. Such devices comprise a container for the liquid dosage form and a mechanism for precisely metering the amount to be administered, and a replaceable injection needle that is changed after each dose. Such devices are often long cylindrical or elliptical shaped devices that are referred to commonly as pen injectors. Various designs and stabilized formulations are known in the art for making and using these metered injection devices, such as for example U.S. RE 041956, EP1003581, U.S. Pat. No. 5,947,934, U.S. Pat. No. 6,582,404, WO93/07922, U.S. Pat. No. 7,686,786 and U.S. Pat. No. 6,899,699. These metered dose administration devices allow patients to dose themselves reliably and at appropriate times given their portability.

In some embodiments, the solid dosage forms disclosed herein are in the form of a tablet, (including a suspension tablet, a fast-melt tablet, a bite-disintegration tablet, a rapid-disintegration tablet, an effervescent tablet, or a caplet), a pill, a powder (including a sterile packaged powder, a dispensable powder, or an effervescent powder) a capsule (including both soft or hard capsules, e.g., capsules made from animal-derived gelatin or plant-derived HPMC, or “sprinkle capsules”), solid dispersion, solid solution, bioerodible dosage form, controlled release formulations, pulsatile release dosage forms, multiparticulate dosage forms, pellets, granules, or an aerosol. In other embodiments, the pharmaceutical formulation is in the form of a powder. In still other embodiments, the pharmaceutical formulation is in the form of a tablet, including but not limited to, a fast-melt tablet. Additionally, pharmaceutical formulations of an ACTH peptide or fragment, analog, complex or aggregate thereof, or any combination thereof, are optionally administered as a single capsule or in multiple capsule dosage form. In some embodiments, the pharmaceutical formulation is administered in two, or three, or four, capsules or tablets.

In another aspect, dosage forms include microencapsulated formulations. In some embodiments, one or more other compatible materials are present in the microencapsulation material. Exemplary materials include, but are not limited to, pH modifiers, erosion facilitators, anti-foaming agents, antioxidants, flavoring agents, and carrier materials such as binders, suspending agents, disintegration agents, filling agents, surfactants, solubilizers, stabilizers, lubricants, wetting agents, and diluents.

Exemplary microencapsulation materials useful for delaying the release of the formulations including an ACTH peptide or fragment, analog, complex or aggregate thereof, or any combination thereof, include, but are not limited to, hydroxypropyl cellulose ethers (HPC) such as Klucel® or Nisso HPC, low-substituted hydroxypropyl cellulose ethers (L-HPC), hydroxypropyl methyl cellulose ethers (HPMC) such as Seppifilm-LC, Pharmacoat®, Metolose SR, Methocel®-E, Opadry YS, PrimaFlo, Benecel MP824, and Benecel MP843, methylcellulose polymers such as Methocel®-A, hydroxypropylmethylcellulose acetate stearate Aqoat (HF-LS, HF-LG, HF-MS) and Metolose®, Ethylcelluloses (EC) and mixtures thereof such as E461, Ethocel®, Aqualon®-EC, Surelease®, Polyvinyl alcohol (PVA) such as Opadry AMB, hydroxyethylcelluloses such as Natrosol®, carboxymethylcelluloses and salts of carboxymethylcelluloses (CMC) such as Aqualon®-CMC, polyvinyl alcohol and polyethylene glycol co-polymers such as Kollicoat IR®, monoglycerides (Myverol), triglycerides (KLX), polyethylene glycols, modified food starch, acrylic polymers and mixtures of acrylic polymers with cellulose ethers such as Eudragit® EPO, Eudragit® L30D-55, Eudragit® FS 30D Eudragit® L100-55, Eudragit® L100, Eudragit® 5100, Eudragit® RD100, Eudragit® E100, Eudragit® L12.5, Eudragit® 512.5, Eudragit® NE30D, and Eudragit® NE 40D, cellulose acetate phthalate, sepifilms such as mixtures of HPMC and stearic acid, cyclodextrins, and mixtures of these materials.

The pharmaceutical solid oral dosage forms including formulations described herein, which include an ACTH peptide or fragment, analog, complex or aggregate thereof, or any combination thereof, are optionally further formulated to provide a controlled release of the ACTH peptide or fragment, analog, complex or aggregate thereof, or any combination thereof. Controlled release refers to the release of the ACTH peptide or fragment, analog, complex or aggregate thereof, or any combination thereof, from a dosage form in which it is incorporated according to a desired profile over an extended period of time. Controlled release profiles include, for example, sustained release, prolonged release, pulsatile release, and delayed release profiles. In contrast to immediate release compositions, controlled release compositions allow delivery of an agent to a individual over an extended period of time according to a predetermined profile. Such release rates provide levels of ACTH peptide or fragment, analog, complex or aggregate thereof, or any combination thereof, for an extended period of time and thereby provide a longer period of pharmacologic response while minimizing side effects as compared to conventional rapid release dosage forms. Such longer periods of response provide for many inherent benefits that are not achieved with the corresponding short acting, immediate release preparations, such as reduction of side effects, by blunting maximum blood levels (Cmax) which are often high in immediate release formulations. Examples of such extended release dosage forms suitable for use with the ACTH peptide or fragments, analogs, complexes or aggregates thereof include, but are not limited to, for example, US published application 20120225816. In some embodiments, the formulations described herein, which include an ACTH peptide or fragment, analog, complex or aggregate thereof, or any combination thereof, are delivered using an implant. In some embodiments, the implants are formulated to provide a controlled release of the ACTH peptide or fragment, analog, complex or aggregate thereof, or any combination thereof. In some embodiments, the implant is an erodible implant.

In other embodiments, the formulations described herein, which include an ACTH peptide or fragment, analog, complex or aggregate thereof, or any combination thereof, are delivered using a pulsatile dosage form. A pulsatile dosage form is capable of providing one or more immediate release pulses at predetermined time points after a controlled lag time or at specific sites. Pulsatile dosage forms including the formulations described herein, which include an ACTH peptide or fragment, analog, complex or aggregate thereof, or any combination thereof, are optionally administered using a variety of pulsatile formulations that include, but are not limited to, those described in U.S. Pat. Nos. 5,011,692, 5,017,381, 5,229,135, and 5,840,329. Other pulsatile release dosage forms suitable for use with the present formulations include, but are not limited to, for example, U.S. Pat. Nos. 4,871,549, 5,260,068, 5,260,069, 5,508,040, 5,567,441 and 5,837,284.

Liquid formulation dosage forms for oral administration are optionally aqueous suspensions selected from the group including, but not limited to, pharmaceutically acceptable aqueous oral dispersions, emulsions, solutions, elixirs, gels, and syrups. See, e.g., Singh et al., Encyclopedia of Pharmaceutical Technology, 2nd Ed., pp. 754-757 (2002). In addition to the ACTH peptide or fragment, analog, complex or aggregate thereof, or any combination thereof, the liquid dosage forms optionally include additives, such as: (a) disintegrating agents; (b) dispersing agents; (c) wetting agents; (d) at least one preservative, (e) viscosity enhancing agents, (f) at least one sweetening agent, and (g) at least one flavoring agent. In some embodiments, the aqueous dispersions further includes a crystal-forming inhibitor.

Suitable intranasal formulations include those described in, for example, U.S. Pat. Nos. 4,476,116, 5,116,817 and 6,391,452. Nasal dosage forms generally contain large amounts of water in addition to the active ingredient. Minor amounts of other ingredients such as pH adjusters, emulsifiers or dispersing agents, preservatives, surfactants, gelling agents, or buffering and other stabilizing and solubilizing agents are optionally present.

For administration by inhalation, the ACTH peptide or fragment, analog, complex or aggregate thereof, or any combination thereof, is optionally in a form as an aerosol, a mist or a fine-particulate powder. Pharmaceutical compositions described herein are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol, the dosage unit is determined by providing a valve to deliver a metered amount. Capsules and cartridges of, such as, by way of example only, gelatin for use in an inhaler or insufflator are formulated containing a powder mix of the ACTH peptide or fragment, analog, complex or aggregate thereof, or any combination thereof, and a suitable powder base such as lactose or starch. Non-limiting examples of powder formulations and inhalation devices are described in WO 91/16038, WO 2014/12069, U.S. Pat. No. 8,633,152 and U.S. Pat. No. 8,636,001.

Buccal formulations that include an ACTH peptide or fragment, analog, complex or aggregate thereof, or any combination thereof, include, but are not limited to, U.S. Pat. Nos. 4,229,447, 4,596,795, 4,755,386, and 5,739,136. In addition, the buccal dosage forms described herein optionally further include a bioerodible (hydrolysable) polymeric carrier that also serves to adhere the dosage form to the buccal mucosa. The buccal dosage form is fabricated so as to erode gradually over a predetermined time period, wherein the delivery of the ACTH peptide or fragment, analog, complex or aggregate thereof, or any combination thereof, is provided essentially throughout. Buccal drug delivery avoids the disadvantages encountered with oral drug administration, e.g., slow absorption, degradation of the active agent by fluids present in the gastrointestinal tract and/or first-pass inactivation in the liver. The bioerodible (hydrolysable) polymeric carrier generally comprises hydrophilic (water-soluble and water-swellable) polymers that adhere to the wet surface of the buccal mucosa. Examples of polymeric carriers useful herein include acrylic acid polymers and co, e.g., those known as “carbomers” (Carbopol®, which may be obtained from B.F. Goodrich, is one such polymer). Other components also be incorporated into the buccal dosage forms described herein include, but are not limited to, disintegrants, diluents, binders, lubricants, flavoring, colorants, preservatives, and the like. For buccal or sublingual administration, the compositions optionally take the form of tablets, lozenges, or gels formulated in a conventional manner.

Transdermal formulations of an ACTH peptide or fragment, analog, complex or aggregate thereof, or any combination thereof, are administered for example by those described in U.S. Pat. Nos. 3,598,122, 3,598,123, 3,710,795, 3,731,683, 3,742,951, 3,814,097, 3,921,636, 3,972,995, 3,993,072, 3,993,073, 3,996,934, 4,031,894, 4,060,084, 4,069,307, 4,077,407, 4,201,211, 4,230,105, 4,292,299, 4,292,303, 5,336,168, 5,665,378, 5,837,280, 5,869,090, 6,923,983, 6,929,801 and 6,946,144.

The transdermal formulations described herein include at least three components: (1) a formulation of an ACTH peptide or fragment, analog, complex or aggregate thereof, or any combination thereof; (2) a penetration enhancer; and (3) an aqueous adjuvant. In addition, transdermal formulations include components such as, but not limited to, gelling agents, creams and ointment bases, and the like. In some embodiments, the transdermal formulation further includes a woven or non-woven backing material to enhance absorption and prevent the removal of the transdermal formulation from the skin. In other embodiments, the transdermal formulations described herein maintain a saturated or supersaturated state to promote diffusion into the skin.

In some embodiments, formulations suitable for transdermal administration of an ACTH peptide or fragment, analog, complex or aggregate thereof, or any combination thereof, employ transdermal delivery devices and transdermal delivery patches and are lipophilic emulsions or buffered, aqueous solutions, dissolved and/or dispersed in a polymer or an adhesive. Such patches are optionally constructed for continuous, pulsatile, or on demand delivery of pharmaceutical agents. Still further, transdermal delivery of the ACTH peptide or fragment, analog, complex or aggregate thereof, or any combination thereof, is optionally accomplished by means of iontophoretic patches and the like. Additionally, transdermal patches provide controlled delivery of the ACTH peptide or fragment, analog, complex or aggregate thereof, or any combination thereof. The rate of absorption is optionally slowed by using rate-controlling membranes or by trapping the ACTH peptide or fragment, analog, complex or aggregate thereof, or any combination thereof, within a polymer matrix or gel. Conversely, absorption enhancers are used to increase absorption. An absorption enhancer or carrier includes absorbable pharmaceutically acceptable solvents to assist passage through the skin. For example, transdermal devices are in the form of a bandage comprising a backing member, a reservoir containing the ACTH peptide or fragment, analog, complex or aggregate thereof, or any combination thereof, optionally with carriers, optionally a rate controlling barrier to deliver the ACTH peptide or fragment, analog, complex or aggregate thereof, or any combination thereof, to the skin of the host at a controlled and predetermined rate over a prolonged period of time, and means to secure the device to the skin.

Formulations that include an ACTH peptide or fragment, analog, complex or aggregate thereof, or any combination thereof, suitable for intramuscular, intrathecal, subcutaneous, or intravenous injection include physiologically acceptable sterile, pyrogen-free aqueous or non-aqueous solutions, dispersions, suspensions or emulsions, and sterile powders for reconstitution into sterile injectable solutions or dispersions. Examples of suitable aqueous and non-aqueous carriers, diluents, solvents, or vehicles including sterile, pyrogen-free water, ethanol, polyols (propyleneglycol, polyethylene-glycol, glycerol, cremophor and the like), suitable mixtures thereof, vegetable oils (such as olive oil) and injectable organic esters such as ethyl oleate. Gelling agents such as gelatin or long-chain fatty acid salts, such as aluminum mono-, di- or tri-stearate, aluminum tristearate or laurate, myristate, palmitate, and oleate salts may provide a prolonged release of active agents after intramuscular or subcutaneous injection. Preservatives such as phenolic compounds (phenol, m-cresol, and benzyl alcohol may be added) and antioxidants such as cysteine, ascorbic acid or BHT may also be present. Proper fluidity is maintained, for example, by the use of a coating such as lecithin, by the maintenance of the required particle size in the case of dispersions, and by the use of surfactants. Formulations suitable for subcutaneous injection also contain optional additives such as wetting, emulsifying, osmolarity-adjusting, pH-adjusting and dispensing agents.

For intravenous injections, an ACTH peptide or fragment, analog, complex or aggregate thereof, or any combination thereof, is optionally formulated in aqueous solutions, preferably in physiologically compatible buffers such as Hank's solution, Ringer's solution, or physiological saline buffer. For transmucosal administration, penetrants appropriate to the barrier to be permeated are used in the formulation. For other parenteral injections including intrathecal and intramuscular injections, appropriate formulations include aqueous or nonaqueous solutions, preferably with physiologically compatible buffers or excipients.

Parenteral injections optionally involve bolus injection or continuous infusion. Formulations for injection are optionally presented in unit dosage form, e.g., in ampoules or in multi dose containers, with an added preservative. In some embodiments, the pharmaceutical composition described herein are in a form suitable for parenteral injection as a sterile suspensions, solutions or emulsions in oily or aqueous vehicles, and contain formulatory agents such as suspending, stabilizing and/or dispersing agents. Pharmaceutical formulations for parenteral administration include aqueous solutions of the ACTH peptide or fragment, analog, complex or aggregate thereof, or any combination thereof, in water soluble form. Additionally, suspensions of the ACTH peptide or fragment, analog, complex or aggregate thereof, or any combination thereof, are optionally prepared as appropriate oily injection suspensions.

In some embodiments, the ACTH peptide or fragment, analog, complex or aggregate thereof, or any combination thereof, is administered topically and formulated into a variety of topically administrable compositions, such as solutions, suspensions, lotions, gels, pastes, medicated sticks, balms, creams or ointments. Such pharmaceutical compositions optionally contain solubilizers, stabilizers, tonicity enhancing agents, buffers and preservatives.

The ACTH peptide or fragment, analog, complex or aggregate thereof, or any combination thereof, is also optionally formulated in rectal compositions such as enemas, rectal gels, rectal foams, rectal aerosols, suppositories, jelly suppositories, or retention enemas, containing conventional suppository bases such as cocoa butter or other glycerides, as well as synthetic polymers such as polyvinylpyrrolidone, PEG, and the like. In suppository forms of the compositions, a low-melting wax such as, but not limited to, a mixture of fatty acid glycerides, optionally in combination with cocoa butter is first melted.

### EXAMPLES

### Example 1

### Clinical Study of Patients for Acthar Treatment of Intractable Chronic Migraine

The purpose of this study is to evaluate the efficacy of ACTH gel (Acthar) the treatment of chronic migraine headache in chronic migraine patients who have failed multiple treatments, including onabotulinumtoxin A (Botox®, which is indicated for prophylaxis of headaches in adult patients with chronic migraine (≧15 days per month with headache lasting 4 hours a day or longer) Failure of previous treatments is defined in this study as having a less than 30% reduction from baseline in the number of headache days per month. The number of headache days after 30 days of Acthar treatment is compared to baseline.

Research Design and Methods:

Study Type: Interventional

Study Design:

Allocation: Randomized

Endpoint Classification: Efficacy

Intervention Model: Parallel Arms

Masking: Open Label Adaptive

Primary Purpose: Treatment

Study Outcome Measures:

The primary outcome measure is the comparison of the total number of headache days after 30 days of treatment to the number of headache days during the baseline 30 day screening. The average number of headache days across all treated patients (40 and 80 IU) is compared to the average number of headache days during baseline screening.

Secondary outcome measures include comparing the total number of headache days after 30 days of treatment to the total number of headache days during the 30 day baseline screening period for all treated patients (40 and 80 IU). Also, a hierarchical analysis of the number of headache and (separately) migraine days subjects on 40 IU experience on average during the 30 day treatment period compared to the number they experienced on average during the baseline screening period is performed. If statistical significance (p<0.05) is reached, then the 80 IU subjects are analyzed similarly. If a subject drops from the 80 IU group to the 40 IU group due to the defined tolerability criteria, they are included in the 40 IU group for statistical analysis.

Subjects are followed for 6 months after their 30 day treatment of Acthar.

Additional secondary endpoints examined are: change from baseline in the mean number of days of migraine specific acute medication intake; the percentage of patients with greater than 50% reduction in the average number of monthly headache and (separately) migraine days during follow up compared to their recorded historical average and baseline; and change from baseline in the mean severity score of migraines.

Study Procedure:

All patients have a complete history and neurologic examination before treatment and the procedures and potential side effects are explained to the patient. Before inclusion in the study, patients are required to have baseline blood urea nitrogen (BUN)/creatinine blood work, EKG, urinalysis, in addition to pregnancy testing at the beginning and ending of treatment period. During treatment phase, subjects have blood glucose monitored weekly.

This is a pilot study involving 30 subjects, which is a single group pretest and posttest design. Subjects are classified as chronic migraine sufferers according to the ICHD-2R criteria (must demonstrate an average of >=15 headache days per month, of which 8 must be migraines or >=8 days migraine-specific acute medication for at least 3 months prior to the study). Subjects must then demonstrate at least 8 migraine days for the 30-day baseline phase measured by a 30-day diary, which is distributed to the subject at their screening visit.

Subjects are screened in and report to the clinic (defined as day 0) and receive injection training, their first injection, study materials and medications and headache diaries.

After the 30 day baseline screen, subjects are randomized to one of two arms. The first arm is treatment with 80 IU of Acthar injected subcutaneously for 30 days. First, Acthar is injected once a day for 5 days. On weeks 2, 3 and 4, subjects inject 80 IU of Acthar subcutaneously 3 times during each week, every other day. For example, injections are on Monday, Wednesday and Friday of each treatment week.

In the second arm of the study, subjects are injected with 40 IU of Acthar subcutaneously for 30 days. In the first week Acthar is injected once a day for 5 days. On weeks 2, 3, and 4, subjects inject 40 IU of Acthar subcutaneously for 3 days. Once again, injections are given every other day during treatment weeks 2, 3, and 4. Patients enrolled in the 80 IU study arm have the option to be moved to the 40 IU study arm if they have tolerability issues with the higher dose, which may include, but are not limited to, the following: blood pressure fluctuations, fasting blood glucose fluctuations, weight gain and edema.

Subjects are followed for 24 weeks post treatment with monthly office visits at which time safety assessments, medication dispensing and adherence monitoring are performed (a detailed study visit timeline will be prepared detailing each measure). During the entire study, subjects keep a headache diary, provided by the investigator, documenting severity and frequency of headaches. Severity is rated on a 10 point pain scale with 10/10 being the most severe. Frequency is determined by the number of days a subject experiences headache that lasts beyond 4 hours and meets the IHS criteria for migraine.

Criteria:

Inclusion Criteria:

Subject is male or female; 18-60 years of age; has a history of chronic migraine as classified by the International Headache Classification, ICHD-2R (i.e. must demonstrate an average of >=15 headache days per month, of which >=8 must be migraine days or >=8 days of migraine-specific acute medication-ergotamine or triptans for at least 3 months prior to study; demonstrates at least >=8 migraine days or >=8 days of migraine specific acute medications—ergotamine or triptans during 30 day baseline screening; is able to differentiate migraine from any other headache they may experience (e.g., tension-type headache); has a previous history of failure of prophylaxis treatment of migraine which can include anti-seizure medications and/or TCA's prescribed for the treatment of chronic migraine; did not have botulinum toxin within 4 months before study enrollment.

Subject is considered a non-responder to previous treatment with Botox. Botox failure will be defined by previous documentation (at the discretion of the PI) or as having less than 30% reduction of headache days per month on Botox.

If female of childbearing potential, has a negative urine pregnancy test at Visits 1 and 7, and uses, or agrees to use, for the duration of the study, a medically acceptable form of contraception as determined by the investigator.

Medically acceptable forms of contraception as determined by the investigator are complete abstinence from intercourse from 2 weeks prior to administration of study drug throughout the study, and for a time interval after completion or premature discontinuation from the study to account for elimination of the study drug (a minimum of 7 days); or, surgically sterile (hysterectomy or tubal ligation or otherwise incapable of pregnancy); or, sterilization of male partner; or, intrauterine device with published data showing lowest expected failure rate is less than 1% per year; or, double barrier method (i.e., 2 physical barriers OR 1 physical barrier plus spermicide) for a least 1 month prior to Visit 1 and throughout study; or, hormonal contraceptives for at least 3 months prior to Visit 1 and throughout study.

Exclusion Criteria:

Subject is unable to understand the study requirements, the informed consent, or complete headache records as required per protocol; or is pregnant, actively trying to become pregnant, or breast-feeding; or has a significant systemic disease that is equally painful or more painful than migraine, or has a progressive neurological disorder such as MS; or has a history of chronic disease of the immune system other than MS or a known immunodeficiency syndrome such as HIV; or has sensitivity to proteins of porcine origin; or has a known or ‘new’ diagnosis of diabetes mellitus (if screening blood glucose is suspicious for diabetes [≧126 mg/dL or ≧7 mmol/L if fasting; ≧200 mg/dL or 11.1 mmol/L if random testing] a patient should be further evaluated for diabetes mellitus), or a known or ‘new’ diagnosis of hypothyroidism not adequately controlled with medication; or has previously taken Acthar for any reason; or has any contraindications listed on the Acthar PI, or has a history of cluster headache, chronic tension type headache, or headache due to medication over use according to IHS guidelines, in the 3 months prior to study enrollment or during the baseline phase; or has received any other investigative drug 30 days prior to enrollment in this study, or who in the opinion of the PI, has a condition for which they should not be enrolled in the study.

Safety Evaluations:

Adverse Events:

All adverse events that occur between the first study related treatment and the last study related treatment are reported by the subject (or legal guardian) for the duration of the study.

### Example 2

### Pharmaceutical Compositions

### Example 2a

### Parenteral Composition

To prepare a parenteral pharmaceutical composition suitable for administration by intrathecal or intramuscular or intravenous or subcutaneous injection, 100 mg of a water-soluble salt of ACTH peptide or fragment, analog, complex or aggregate thereof, or any combination thereof, described herein, is dissolved in DMSO and then mixed with 10 mL of 0.9% sterile pyrogen-free saline solution. A preservative and/or a stabilizer is optionally added to the mixture. The mixture is incorporated into a dosage unit form suitable for administration by injection.

### Example 2b

### Inhalation Composition

To prepare a pharmaceutical composition for inhalation delivery, 20 mg of ACTH peptide or fragment, analog, complex or aggregate thereof, or any combination thereof, is mixed with 50 mg of anhydrous citric acid and 100 mL of 0.9% sodium chloride solution. The mixture is incorporated into an inhalation delivery unit, such as a nebulizer, which is suitable for inhalation administration.

### Example 2c

### Rectal Gel Composition

To prepare a pharmaceutical composition for rectal delivery, 100 mg of ACTH peptide or fragment, analog, complex or aggregate thereof, or any combination thereof, is mixed with 2.5 g of methylcelluose (1500 mPa), 100 mg of methylparapen, 5 g of glycerin and 100 mL of purified water. The resulting gel mixture is then incorporated into rectal delivery units, such as syringes, which are suitable for rectal administration.

### Example 2d

### Topical Gel Composition

To prepare a pharmaceutical topical gel composition, 100 mg of ACTH peptide or fragment, analog, complex or aggregate thereof, or any combination thereof, is mixed with 1.75 g of hydroxypropyl cellulose, 10 mL of propylene glycol, 10 mL of isopropyl myristate and 100 mL of purified alcohol USP. The resulting gel mixture is then incorporated into containers, such as tubes, which are suitable for topical administration.

### Example 2e

### Oral Composition

To prepare a pharmaceutical composition for oral delivery, 100 mg of ACTH peptide or fragment, analog, complex or aggregate thereof, or any combination thereof, or a prodrug thereof, is mixed with 750 mg of starch. The mixture is incorporated into an oral dosage unit, such as a hard gelatin capsule, which is suitable for oral administration.

### Example 2f

### Nasal Spray Solution

To prepare a pharmaceutical nasal spray solution, 10 g of ACTH peptide or fragment, analog, complex or aggregate thereof, or any combination thereof, is mixed with 30 mL of a 0.05M phosphate buffer solution (pH 4.4). The solution is placed in a nasal administrator designed to deliver 100 μl of spray for each application.

### REFERENCES

Domenico D'Amico and Stewart J. Tepper. Prophylaxis of migraine: general principles and patient acceptance, Neuropsychiatr Dis Treat. 2008 December; 4(6): 1155-1167.
Brunson K L, Khan N, Eghbal-Ahmadi M, Baram T Z. Corticotropin (ACTH) acts directly on amygdala neurons to down-regulate corticotropin-releasing hormone gene expression. Ann Neurol. 2001; 49:304-312.
Stewart J. Tepper and Mark J. Stillman, Clinical and Preclinical Rationale for CGRP-Receptor Antagonists in the Treatment of Migraine, Headache 2008; 48(8):1259-1268.
Durham P L, Cady R, Cady R. Regulation of calcitonin gene-related peptide secretion from trigeminal nerve cells by botulinum toxin type A: Implications for migraine therapy. Headache. 2004; 44:35-43.
Ulrich-Lai Y M, Harding-Rose C A, Guo A, et al. ACTH inhibits the capsaicin-evoked release of CGRP from rat adrenal afferent nerves. Am J Physiol Regul Integr Comp Physiol 2001; 280(1): R137-142.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1-64. (canceled)

65. A method of treating an individual diagnosed with migraine comprising administration of an adrenocorticotropic hormone (ACTH) peptide, or fragment, analog, complex or aggregate thereof, or any combination thereof, to an individual in need thereof at a therapeutically effective amount and interval.

66. The method of claim 65, wherein the migraine is chronic migraine, migraine with aura, migraine without aura, familial hemiplegic migraine, sporadic hemiplegic migraine, basilar-type migraine, status migrainosus, probable migraine or retinal migraine.

67. The method of claim 65, wherein the treatment is prophylactic and the migraine is chronic migraine.

68. The method of claim 65, wherein the treatment is acute and the migraine is status migrainosus.

69. The method of claim 65, wherein the ACTH peptide or fragment, analog, complex or aggregate thereof, or any combination thereof is administered as an initial phase of from one to five doses at daily intervals and a subsequent phase of one or more doses administered at intervals greater than one day.

70. The method of claim 69, wherein each of the one to five doses of the initial phase is between about 10 IU and about 150 IU, and each of the one or more doses of the subsequent phase is between about 10 IU and about 80 IU.

71. The method of claim 65, wherein the ACTH peptide or fragment, analog, complex or aggregate thereof, or any combination thereof, is an ACTH₁₋₃₉ peptide having the formula:
```
(SEQ ID NO: 1)
H-Ser-Tyr-Ser-Met-Glu-His-Phe-Arg-Trp-Gly-
      1 2 3 4 5 6 7 8 9 10
Lys-Pro-Val-Gly-Lys-Lys-Arg-Arg-Pro-Val-
11 12 13 14 15 16 17 18 19 20
Lys-Val-Tyr-Pro-Asp-Gly-Ala-Glu-Asp-Gln-
  21 22 23 24 25 26 27 28 29 30
Leu-Ala-Glu-Ala-Phe-Pro-Leu-Glu-Phe-OH
  31 32 33 34 35 36 37 38 39
```
or a fragment, complex, aggregate, or analog thereof, or any combination thereof.

72. The method of claim 65, wherein the ACTH peptide or fragment, analog, complex or aggregate thereof, or any combination thereof, is an ACTH₁₋₂₄ peptide having the formula:
(SEQ ID NO: 2)
H-Ser-Tyr-Ser-Met-Glu-His-Phe-Arg-Trp-Gly-
1 2 3 4 5 6 7 8 9 10
Lys-Pro-Val-Gly-Lys-Lys-Arg-Arg-Pro-Val-
11 12 13 14 15 16 17 18 19 20
Lys-Val-Tyr-Pro
21 22 23 24
or a fragment, complex, aggregate, or analog thereof, or any combination thereof.

73. The method of claim 65, wherein the ACTH peptide or fragment, analog, complex or aggregate thereof, or any combination thereof, is a porcine-derived pituitary extract.

74. The method of claim 65, wherein the ACTH analog is a peptide with about 50%, 60%, 70%, 80%, 90%, or 100% homology to ACTH.

75. The method of claim 65, wherein the ACTH peptide or fragment, analog, complex or aggregate thereof, or any combination thereof, is formulated as an aerosol, nasal spray, syrup, gel, liquid solution, emulsion, suspension, powder, injectable solution, aqueous liquid dispersion, self-emulsifying dispersion, solid solution, liposomal dispersion, immediate release formulation, prodrug, prolonged release formulation, controlled release formulation, fast melt formulation, delayed release formulation, pulsatile release formulation, multiparticulate formulation or mixed immediate and controlled release formulation.

76. The method of claim 65, wherein the ACTH peptide or fragment, analog, complex or aggregate thereof, or any combination thereof, is administered intramuscularly, subcutaneously, intravenously, systemically, transmucosally, parenterally, intranasally, buccally, or transdermically, rectally, by inhalation or by implant.

77. The method of claim 65, wherein the ACTH peptide or fragment, analog, complex or aggregate thereof, or any combination thereof, is a porcine-derived ACTH peptide or fragment, or any combination thereof, formulated in a prolonged release gel.

78. The method of claim 65, wherein the ACTH peptide or fragment, analog, complex or aggregate thereof, or any combination thereof, is a recombinant human ACTH peptide or fragment, analog, complex or aggregate thereof, or any combination thereof.

79. The method of claim 65, further comprising administration of a second therapeutic agent.

80. The method of claim 79, wherein the second therapeutic agent selected from the group consisting of an antiepileptic, topiramate, gabapentin, pregabalin, valproate sodium, and zonisamide; onabotulinumtoxin A, botulinum toxin B, tizanidine, a tricyclic antidepressant, amitriptyline, imipramine, nortriptyline, protriptyline, doxepin, fluoxetine, memantine, triptan, sumatriptan, frovatriptan, almotriptan, rizatriptan, zolmitriptan; dihydroergotamine, metoclopramide, hydroxyzine, an ergot derivative, methysergide, methylergonovine, methylergometrine, a neuroleptic, prochlorperazine, chlorpromazine, droperidol, a corticosteroid, magnesium, a NSAID, ketorolac, naproxen sodium, acetaminophen, an opioid, an antiemetic, metoclopramide, ondansetron, vitamin B12, timolol and propranolol.

81. The method of claim 65 wherein the individual's migraine is refractory to other migraine treatments.

82. The method of claim 81 wherein the individual's migraine is refractory to onabotulinum toxin A (Botox®) treatment.

83. The method of claim 65 wherein the individual is contraindicated for treatment with another migraine medication.

84. The method of claim 83 wherein the contraindicated treatment is a triptan, an ergot derivative, dihydroergotamine, methysergide, methylergonovine or methylergometrine.


SEQUENCE LISTING (8 sequences)

SEQ ID NO: 1 - protein, 39 aa, Artificial Sequence
  synthetic peptide
SYSMEHFRWGKPVGKKRRPVKVYPDGAEDQLAEAFPLEF

SEQ ID NO: 2 - protein, 24 aa, Artificial Sequence
  synthetic peptide
SYSMEHFRWGKPVGKKRRPVKVYP

SEQ ID NO: 3 - protein, 17 aa, Artificial Sequence
  synthetic peptide
SYSMEHFRWGKPVGKKR

SEQ ID NO: 4 - protein, 7 aa, Artificial Sequence
  synthetic peptide
MEHFRWG

SEQ ID NO: 5 - protein, 6 aa, Artificial Sequence
  synthetic peptide
MEHFKF

SEQ ID NO: 6 - protein, 13 aa, Artificial Sequence
  synthetic peptide
SYSMEHFRWGKPV

SEQ ID NO: 7 - protein, 13 aa, Artificial Sequence
  synthetic peptide
SYSMEHFRWGKPV

SEQ ID NO: 8 - protein, 7 aa, Artificial Sequence
  synthetic peptide
AQYFRWG